SEQUENCE LISTING (55 sequences)

SEQ ID NO: 1 - protein, 136 aa, Arabidopsis thaliana
MARTKQSARKSHGGKAPTKQLATKAARKSAPTTGGVKKPHRFRPGTVALREIRKYQKSTELLNRKLPFQRLVREIAQDFKTDLRFQSHAVLALQEAAEAYLVGLFEDTNLCAIHAKRVTIMPKDVQLARRIRAERA

SEQ ID NO: 2 - protein, 136 aa, H.sapiens
MARTKQTARKSTGGKAPRKQLATKAARKSAPSTGGVKKPHRYRPGTVALREIRRYQKSTELLIRKLPFQRLVREIAQDFKTDLRFQSAAIGALQEASEAYLVGLFEDTNLCAIHAKRVTIMPKDIQLARRIRGERA

SEQ ID NO: 3 - protein, 125 aa, Physcomitrella patens
MARRKTTPVHGNHRASTSSVGGAAVRPRKPHRWRPGTKALQEIRHYQKTCDLLIPRLPFARYVKEITMMYASDVSRWTAEALTALQEATEDYMCHLFEDTNLCAIHAKRVTIMPKDLQLARRLRG

SEQ ID NO: 4 - protein, 164 aa, Pinus taeda
MVRRKTVPPRKKSGSGNAASTSGVGVSTPGSAGERGERRGSARLASTPGSDASPSAPSGRKPHRFRPGTVALREIKRYQKSFELLIPSLPFARIVRELTMYYSQVVSRWAAEALVALQEAAEDYIVHLFEDTNLCAIHAKRVTIMPRDLRLARRLRGGGLDRPW

SEQ ID NO: 5 - protein, 177 aa, Boechera holboelli
MARTKHLATRSRPRNQTDATASSSQAAGPSTNPTTRGSEGEDAAQETTPTTSPATGRKKGAKRARYARPQGSQKKPYRYKPGTVALREIRYFQKSINLLIPAASFIRQVRSITHALAPPQITRWTAEALVALQEAAEDYLVGLFSDSMLCAIHARRVTLMRKDFELARRLGGKGRPW

SEQ ID NO: 6 - protein, 177 aa, Boechera stricta
MARTKHLATRSRPRNWTDATASSSQAAGPTTNPTTRGSEGEDAAQEPTPTTSPATGRKKGAKRARYARPQGSQKKPYRYKPGTVALREIRHFQKSINLLIPAASFIRQVRSITHALAPPQITRWTAEALVALQEAAEDYLVGLFSDSMLCAIHARRITLMRKDFELARRLGGKGRPW

SEQ ID NO: 7 - protein, 180 aa, Lepidium virginicum
MARTKRYASRPQRPRNQTDVTVPSSPAAGPSTNPTRRDSEGEGGDDAQQTVPTTSPASISKKASKKNRKATPQSSKKKTYRYKPGTVALREIRHFQKSTHLLIPAAAFIREVRCITQAVAPPQISRWTAEALVALQEAAEDYVVGLLSDSMLCAIHARRVTLMRKDFELARRLGGKGRPW

SEQ ID NO: 8 - protein, 172 aa, Cardaminopsis flexuosa
MARTKHFPNRTRPRNQTDATTPAAGPSTRTTRANQGEETQQTNPTTSPATSKKKGAKRTRRDMPQGSQKKPYRYKPGTVALREIRHFQKSTNLLIPAASFIRQVRSITQMYAPPQINRWTAEALVALQEAAEDYLVGLFSDSMLCAIHARRVTLMRKDFELARRLGGKGRPW

SEQ ID NO: 9 - protein, 139 aa, Hordeum vulgare
MARTKKTVAAKEKRPPCSKSEPQSQPKKKEKRAYRFRPGTVALREIRKYRKSTNMLIPFAPFVRLVRDIADNLTPLSNKKESKPTPWTPLALLSLQESAEYHLVDLFGKANLCAIHSHRVTIMLKDMQLARRIGTRSLW

SEQ ID NO: 10 - protein, 178 aa, Arabidopsis thaliana
MARTKHRVTRSQPRNQTDAAGASSSQAAGPTTTPTRRGGEGGDNTQQTNPTTSPATGTRRGAKRSRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 11 - protein, 152 aa, Populus trichocarpa
MARTKHPVARKRARSPKRSDASPSTPRTPTSSRTRPQANGQQGSSTQRQRKKHRFRSGTVALREIRQYQKTWRPLIPAASFIRCVRMITQEFSREVNRWTAEALVAIQEAAEDFLVHLFEDGMLCAIHAKRVTLMKKDFELARRLGGKGRPW

SEQ ID NO: 12 - protein, 166 aa, Triticum aestivum
MARTKHPAVRKTKALPKKQLGTRPSAGTPRRQETDGAGTSATPRRAGRAAAPGAAEGATGQPKQRKPHRFRPGTVALREIRKYQKSVDFLIPFAPFVRLIKEVTDFFCPEISRWTPQALVAIQEAAEYHLVDVFERANHCAIHAKRVTVMQKDIQLARRIGGRRLW

SEQ ID NO: 13 - protein, 170 aa, Oryza sativa
MARTKHPAVRKSKAEPKKKLQFERSPRPSKAQRAGGGTGTSATTRSAAGTSASGTPRQQTKQRKPHRFRPGTVALREIRKFQKTTELLIPFAPFSRLVREITDFYSKDVSRWTLEALLALQEAAEYHLVDIFEVSNLCAIHAKRVTIMQKDMQLARRIGGRRPWNLNSLR

SEQ ID NO: 14 - protein, 167 aa, Luzula nivea
MARTKHFPQCSRHPKKQRTAAGEAGSSVIAKQNAPAKTGNASSITNSTPARSLKKNKASKRGEKTQAKQRKMYRYRPGTVALREIRKLQKTTDLLVPKASFARLVKEITFQSSKEVNRWQAEALIALQEASECFLVNLLESANMLAIHARRVTIMKKDIQLARRIGA

SEQ ID NO: 15 - protein, 176 aa, Arabidopsis arenosa
MARTKHFATRTGSGNRTDANASSSQAAGPTTTPTTRGTEGGDNTQQTNPTTSPATGGRRPRRARQAMPRGSQKKPYRYKPGTVALREIRHFQKQTNLLIPAASFIRQVRSITHALAPPQINRWTAEALVALQEAAEDYLIGLFSDSMLCAIHARRVTLMRKDFELARRLGGKGRPW

SEQ ID NO: 16 - protein, 157 aa, Zea mays
MARTKHQAVRKTAEKPKKKLQFERSGGASTSATPERAAGTGGRAASGGDSVKKTKPRHRWRPGTVALREIRKYQKSTEPLIPFAPFVRVVRELTNFVTNGKVERYTAEALLALQEAAEFHLIELFEMANLCAIHAKRVTIMQKDIQLARRIGGRRWA

SEQ ID NO: 17 - protein, 154 aa, Sorghum bicolor
MARTKHQAVRKLPQKPKKKLQFERAGGASTSATPRRNAGTGGGAAARGEDLFKKHRWRAGTVALREIRKYQKSTEPLIPFAPFVRVVKELTAFITDWRIGRYTPEALLALQEAAEFHLIELFEVANLCAIHAKRVTVMQKDIQLARRIGGRRWS

SEQ ID NO: 18 - protein, 150 aa, Cichorium intybus
MARTKQPAKRSWGNRKSSQSRASTSTSTSTPRKSPRKDPGRTGERRQQKPHRFKPGAQALREIRRLQKTVNLLIPAAPFIRTVKEISNYIAPEVTRWQAEAIQALQEAAEDYLVQLFEDSMLCSIHAKRVTLMKKDWELARRLTKKGQPW

SEQ ID NO: 19 - protein, 153 aa, Cycas rumphii
MARKKASTPRKKTGTAASTSAVESPPSGVNQTARARRSVGGVAPGAPRTPQASTNVGTPRRPHRFRPGTVALREIRRYQKSFELLIPALPFARNVRELTLHHSREVHRWTAEALVALQEAAEDYIVHLFEDTNLCAIHAKRVTIMPKDMHLAR

SEQ ID NO: 20 - protein, 154 aa, Allium cepa
MARTKQMAHKKLRRKLNVDEAGPSTPVTRSTEVNPKSSRPTPITEDRGTGARKKHRFRPGTVALREIRKYQKTAELLIPAAPFIRLVREITNLYSKEVTRWTPEALLAIQEAAEFFIINLLEEANLCAIHAKRVTLMQKDIQLARRIGGARHFS

SEQ ID NO: 21 - protein, 199 aa, Malus domestica
MARIKHTAHKKSVARKSSTPKEAAAGTGGTSAASPAKQPEPSAPWRRSERSSQRTSESQEQQEPETNAQATPQSKKQKQSERNPQTPQSKKQKPSERNPPPTQKKKWRYRPGTVALREIRYYQKTWNLIIPAAPFIRTVREISINMSKDPVRWTPEALQAIQEAAEDFLVRLFEDSMLCAIHARRVTLMKKDLELARRI

SEQ ID NO: 22 - protein, 150 aa, Lactuta sativa
MARTKQPAKRSWGKRQSAGASTSTSTSTPRKSPRKDPGSSGTGQRQKQKPHRFKPGTQALREIRRLQKTVNLLIPAAPFIRTVKEISNYIAPEVTRWQAEALQALQEAAEDYIVQLFEDSMLCSIHAKRVTLMKKDMELARRLTKKGQPW

SEQ ID NO: 23 - protein, 145 aa, Carthamus tinctorius
MARTKQPAKRSSGKRDARPSTSTPTPRPSARKNPESSGAGDGQRRHRYRPGTQALREIRRLQKTVNLLIPAAPFIRTVKEISNYIAPEVTRWQAEALQALQEAAEDYLIQLFEDSMLCAIHAKRVTLMKKDWELARRLGKKGQPW

SEQ ID NO: 24 - protein, 145 aa, Helianthus exilis; identical to SEQ ID NO: 23

SEQ ID NO: 25 - protein, 150 aa, Gossypium hirsutum
MSRTKHTAAKKPRRKPSAAAAASPATASPHTRSVTAKKTGGPATPTPGKSKRPHRFRAGTRALQEIRKYQKTSNLLVPAASFIREVRAISYRFAPDINRWQAEALVAIQEAEDYLIQLFGDAMLCAIHAKRVTLMKKDIQLARRLGGMGQ

SEQ ID NO: 26 - protein, 155 aa, Glycine max
MARVKHTPASRKSAKKQAPRASTSTQPPPQSQSPATRERRRAQQVEPQQEPEAQGRKKRRNRSGTVALREIRHFQRSCELLIPAAPFIRCVKQITNQFSTEVSRWTPEAVVALQEAAEEYLVHLFEDGMLCAIHARRITLMKKDIELARRLGGIG

SEQ ID NO: 27 - protein, 153 aa, Cucumis melo
MARARHPVQRKSNRTSSGSGAALSPPAVPSTPLNGRTQNVRKAQSPPSRTKKKIRFRPGTVALREIRNLQKSWNLLIPASCFIRAVKEVSNQLAPQITRWQAEALVALQEAAEDFLVHLFEDTMLCAIHAKRVTIMKKDFELARRLGGKGRPW

SEQ ID NO: 28 - protein, 147 aa, Solanum chacoense
MARTKHLAKRSRTKPSVAAGPSATPSTPTRKSPRSAPATSVPKPKQKKRYRPGSVALREIRHFQKTWNLVIPAAPFIRLVREISHFFAPGVTRWQAEALIAIQEAAEDFLVHLFEDAMLCAIHAKRVTLMKKDFELARRLGGKGQPW

SEQ ID NO: 29 - protein, 144 aa, Solanum lycopersicum
MARTKHLAKRSRTTSAAPSATPSTPSRKSPRSAPATSVQKPKQKKRYRPGTVALREIRHFQKTWDLLIPAAPFIRLVREISHFYAPGVTRWQAEALIAIQEAAEDFLVHLFEDAMLCAIHAKRVTLMKKDFELARRLGGKGQPW

SEQ ID NO: 30 - protein, 156 aa, Nicotiana tabacum CENH3-1
MARTKHLALRKQSRPPSRPTATRSAAAAASSAPQSTPTRTSQRTAPSTPGRTQKKKTRYRPGTVALREIRRFQKTWDLLIPAAPFIRLVKEISHFFAPEVTRWQAEALIALQEAAEDFLVHLFDDSMLCAIHAKRVTLMKKDFELARRLGGKARPW

SEQ ID NO: 31 - protein, 120 aa, Nicotiana tabacum
MARTKHLALRKQSRPPSRPTATRSAAAAASSSAPQSTPTRTSQRTAPSTPGRTQKKKTRYRPGTVALREIRRFQKTWNLLIPAAPFIRLVKEISYFFAPEVTRWQAEALIALQEAAEDFL

SEQ ID NO: 32 - protein, 156 aa, Nicotiana tomentosiformis; identical to SEQ ID NO: 30

SEQ ID NO: 33 - protein, 158 aa, Vitis vinifera
MTRTKHLARKSRNRRRQFAATPASPASAGPSSAPPRRPTRTATDASPSTAGSQGQRKPFRYRPGTVALREIRRFQKTTHLLIPAAPFIRTVREISYFFAPEISRWTAEALVALQEAAEDYLVHLFEDAMLCAIHAKRVTLMKKDWELARRIGGKGQPW

SEQ ID NO: 34 - protein, 157 aa, Nicotiana sylvestris
MARTKHLALRKQSRPPSRPTATRSAAAAASSSAPQSTPTRTSQRTAPSTPGRTQKKKTRYRPGTVALREIRRFQKTWNLLIPAAPFIRLVKEISYFFAPEVTRWQAEALIALQEAAEDFLVHLFDDSMLCAIHAKRVTLMKKDFELARRLGGKARPW

SEQ ID NO: 35 - protein, 177 aa, Crucihimalaya himalaica
MARTKHFATRSRPRNQTDATASASQATGPSTNPTTRGSEGEDAARGTNPTTSPATGRKKGVKRARHAMPQGSQKKPYRYKAGTVALREIRHFQKNTNLLIPAASFIRQVKSITYAVAPPQITRWTAEALVALQEAAEDYLVGLFSDSMLCAIHARRVTLMRKDFELARRLGGKGRPW

SEQ ID NO: 36 - protein, 176 aa, Arabidopsis lyrata
MARTKHFATKSRSGNRTDANASSSQAAGPTTTPTTRGTEGGDNTQQTNPTTSPATGGRRPRRARQAMPRVSQNKPYRYKPGTVALREIRHFQKQTNLLIPAASFIRQVRSITHALAPPQINRWTAEALVALQEAAEDYLVGLFSDSMLCAIHARRVTLMRKDFELARRLGGKGRPW

SEQ ID NO: 37 - protein, 170 aa, Capsella bursapastoris
MARTKHFATRSGPRTPAVASSSQAAVPSSSPATRGRVGVDAAAQQPTPATSPATAKKKGAKRARFGRPQGSQKKKPYRYRPGTVALREIRHYQKGTSLLIPAAAFIRQVRSITNAVAPREVNRWTAEALVALQEAAEDFLVGLFSDSMLCAIHARRVTLMRKDFDLARRL

SEQ ID NO: 38 - protein, 178 aa, Raphanus sativus
MARTKHFASRARDRNQPNAAAAAAGPSATPTRRGSSQGEEAQQTTPTTTSPATTASGRKKGTKRTTQAMPKSSKKKTFRYKPGTVALREIRHFQKSTKLLIPSAPFIREVRSITHNLAAAYVTRWTAEALIALQEAAEDFLVGLFSDAMLCAIHAKRVTLMRKDFELARRLGGKGRPF

SEQ ID NO: 39 - protein, 164 aa, Eruca sativa
MARTKHFASRARDRNRNNATASSSAAAAAAGPSATPTRRGSRQGGGGGGGVEAQQGSNKKKKSFRYKPGTVALREIRHFQKTTKLLIPAATFIRLVRSITLDRAKPQVTRWTAEALVALQEAAEDYLVGLFSDSMLCAIHAKRVTLMRKDFELARRLGGKGRPW

SEQ ID NO: 40 - protein, 173 aa, Olimarabidopsis pumila-1
MARTKHNAIRSRDRTGATASSSQAAGPSTNPTAGGSEDAAQQTTPTTSPATGSKKRAKRARQAMPRGSQKKPYRYKPGTVALREIRHFQKTTSLLLPAAPFIRQVRSISSALAPREITRWTAEALVALQEAAEDYLVGLFSDSMLCAIHAKRVTLMRKDFELARRLGGKGRPW

SEQ ID NO: 41 - protein, 174 aa, Olimarabidopsis pumila-2
MTRTKHTVIKSSRPLDRTDASSSQAAGPSTNPTAGSSGDAAQQTTPTTSPATGSTKRAKRARQAMPRGSQKKPYRYKPGTVALREIRHFQKTTSFLIPAAPFIRQVRSISSALAPTQITRWTAEALVALQEAAEDYLVGLFSDSMLCAIHAKRVTLMRKDFELARRLGGKGRPW

SEQ ID NO: 42 - protein, 174 aa, Turritis glabra
MARTKHFATRSRPRNQTDSSSQAAGPSTNPTTGGSEGGDAAQQTTPTTSPATGRKKRAKRAKQAMPQGSQKKPYRYKPGTIALREIRYFQKNTNLLIPAASFIREVRSITHALAPPQISRWTAEALVALQEAAEDYLVGLFSDSMLCAIHARRVTLMRKDFELARRIGGKGRPW

SEQ ID NO: 43 - protein, 176 aa, Arabidopsis halleri-1
MARTKHFAIKSRSGNRTDANASSSQAAGPTTTPTTRGTEGGDNTQQTNPTTSPATGGRRPRRARQAMPRGSQKKPYRYKPGTVALREIRHFQKQTNLLIPAASFIRQVRSITHALAPPQINRWTAEALVALQEAAEDYLVGLFSDSMLCAIHARRVTLMRKDFELTRRLGGKGRPW

SEQ ID NO: 44 - protein, 176 aa, Arabidopsis halleri-2
MARTKHFVTRKGSGNRTDFDANASSSQAAGPTKTPTTRGTEGGDNTQQTTSPATGGRRGPRRARQAMPRGSQKKPYRYKPGTVALREIRHFQKQTNLLIPAASFIRQVRSITHALAPPQINRWTAEALVALQEAAEDYLVGLFSDSMLCAIHARRVTLMRKDFELARRLGGKGRPW

SEQ ID NO: 45 - protein, 175 aa, Arabidopsis lyrata-HTR12A
MARTKHFATRTGSGNRTDANASSSSQAAGPTKTPTTRGTEGGDNTQQTTSPATGGRRGPRRARQAMPRGSQKKPYRYKPGTVALREIRHFQKQTNLLIPAASFIRQARSITHALAPPQINRWTAEALVALQEAAEDYLVGLFSDSMLCAIHARRVTLMRKDFELARRLGGKGRPW

SEQ ID NO: 46 - protein, 172 aa, Arabidopsis lyrata-HTR12B
MARTKHFATKSRTDANASSSQAAGPTTTPTTRGTEGGDNTQQTNPTTSPATGGRRPRRARQAMPRGSQKKPYRYKPGTVALREIRHFQKQTNLLIPAASFIRQVRSITHALAPPQINRWTAEALVALQEAAEDYLVGLFSDSMLCAIHARRVTLMRKDFELARRLGGKGRPW

SEQ ID NO: 47 - protein, 163 aa, Saccharum officinalis
MARTKHQAVRRPTQKPKKKLQFERAGGASTSATPERNAGTGGGAAARVTRGRVEKKHRWRVGTVALREIRKYQKSTEPLIPFAPFVRVVKELTGFITDWRIGRYTPEALLALQEAAEFHLIELFQVANLCAIHAKRVTVMQKDIQLARRIGGKRWAYPFFLPY

SEQ ID NO: 48 - protein, 181 aa, Brassica napa
MARTKHFASRARDRNPTNATASSSAAAAAGPSATPTRRGGSQGGEAQQTTPPATTTAGRKKGGTKRTKQAMPKSSNKKKTFRYKPGTVALREIRHFQKTTKLLIPAASFIREVRSVTQIFAPPDVTRWTAEALMAIQEAAEDFLVGLFSDAMLCAIHARRVTLMRKDFELARRLGGKGRPL

SEQ ID NO: 49 - protein, 180 aa, Lepidium oleraceum
MARTKRFASRPQRPRNQTDTTVPSSPAAGPSTNPTRRDSEGEGGDDAQQTVPTTSPATTSKKVSKRTGKVMPQSSKKKTYRYKPGTVALREIRHFQKSTHFLIPAAAFIREVRCITQAVAPPQISRWTAEALVALQEAAEDYVVGLLSDSMLCAIHARRVTLMRKDFELARRLGGKGRPW

SEQ ID NO: 50 - protein, 182 aa, Brassica rapa
MARTKHFASRARDRNPTNATASSSAAAAAGPSATPTRRGGSQGGEAQQTATPPATTTAGRKKGGTKRTKQAMPKSSNKKKTFRYKPGTVALREIRHFQKTTKLLIPAASFIREVRSVTQIFAPPDVTRWTAEALMAIQEAAEDFLVGLFSDAMLCAIHARRVTLMRKDFELARRLGGKGRPL

SEQ ID NO: 51 - protein, 178 aa, Arabidopsis thaliana
  MOD_RES (82)..(82): Xaa is Pro, Ser or Leu
  MOD_RES (83)..(83): Xaa is Gly, Arg or Glu
  MOD_RES (84)..(84): Xaa is Thr or Ile
  MOD_RES (86)..(86): Xaa is Ala, Thr or Val
  MOD_RES (89)..(89): Xaa is Glu or Lys
  MOD_RES (100)..(100): Xaa is Leu or Phe
  MOD_RES (104)..(104): Xaa is Ala or Val
  MOD_RES (124)..(124): Xaa is Arg, Cys or His
  MOD_RES (127)..(127): Xaa is Ala or Val
  MOD_RES (128)..(128): Xaa is Glu or Lys
  MOD_RES (129)..(129): Xaa is Ala, Thr or Val
  MOD_RES (132)..(132): Xaa is Ala, Thr or Val
  MOD_RES (135)..(135): Xaa is Glu or Lys
  MOD_RES (136)..(136): Xaa is Ala, Thr or Val
  MOD_RES (137)..(137): Xaa is Ala or Val
  MOD_RES (138)..(138): Xaa is Glu or Lys
  MOD_RES (148)..(148): Xaa is Ser or Thr
  MOD_RES (151)..(151): Xaa is Cys or Tyr
  MOD_RES (152)..(152): Xaa is Ala, Thr or Val
  MOD_RES (154)..(154): Xaa is His or Tyr
  MOD_RES (155)..(155): Xaa is Ala, Thr or Val
  MOD_RES (157)..(157): Xaa is Arg, Cys or His
  MOD_RES (158)..(158): Xaa is Val or Ile
  MOD_RES (159)..(159): Xaa is Thr or Ile
  MOD_RES (161)..(161): Xaa is Met or Ile
  MOD_RES (164)..(164): Xaa is Asp or Asn
  MOD_RES (168)..(168): Xaa is Ala, Thr or Val
  MOD_RES (172)..(172): Xaa is Gly, Arg or Glu
  MOD_RES (173)..(173): Xaa is Gly, Arg or Glu
MARTKHRVTRSQPRNQTDAAGASSSQAAGPTTTPTRRGGEGGDNTQQTNPTTSPATGTRRGAKRSRQAMPRGSQKKSYRYRXXXVXLKXIRHFQKQTNLXIPAXSFIREVRSITHMLAPPQINXWTXXXLVXLQXXXXDYLVGLFSDXMLXXIXXRXXXLXRKXFELXRRLXXKGRPW

SEQ ID NO: 52 - protein, 178 aa, Arabidopsis thaliana; identical to SEQ ID NO: 51
  MOD_RES (82)..(84): Xaa is any amino acid
  MOD_RES (86)..(86): Xaa is any amino acid
  MOD_RES (89)..(89): Xaa is any amino acid
  MOD_RES (100)..(100): Xaa is any amino acid
  MOD_RES (102)..(102): Xaa is any amino acid
  MOD_RES (104)..(104): Xaa is any amino acid
  MOD_RES (124)..(124): Xaa is any amino acid
  MOD_RES (127)..(129): Xaa is any amino acid
  MOD_RES (132)..(132): Xaa is any amino acid
  MOD_RES (135)..(138): Xaa is any amino acid
  MOD_RES (148)..(148): Xaa is any amino acid
  MOD_RES (151)..(152): Xaa is any amino acid
  MOD_RES (154)..(155): Xaa is any amino acid
  MOD_RES (157)..(159): Xaa is any amino acid
  MOD_RES (161)..(161): Xaa is any amino acid
  MOD_RES (164)..(164): Xaa is any amino acid
  MOD_RES (168)..(168): Xaa is any amino acid
  MOD_RES (172)..(173): Xaa is any amino acid

SEQ ID NO: 53 - protein, 180 aa, Artificial Sequence
  synthetic polypeptide alignment sequence for CENH3
  misc_feature (7)..(9): Xaa can be any naturally occurring amino acid
  misc_feature (11)..(13): Xaa can be any naturally occurring amino acid
  misc_feature (16)..(16): Xaa can be any naturally occurring amino acid
  misc_feature (19)..(19): Xaa can be any naturally occurring amino acid
  misc_feature (21)..(22): Xaa can be any naturally occurring amino acid
  misc_feature (24)..(24): Xaa can be any naturally occurring amino acid
  misc_feature (26)..(26): Xaa can be any naturally occurring amino acid
  misc_feature (31)..(32): Xaa can be any naturally occurring amino acid
  misc_feature (41)..(41): Xaa can be any naturally occurring amino acid
  misc_feature (43)..(45): Xaa can be any naturally occurring amino acid
  misc_feature (47)..(47): Xaa can be any naturally occurring amino acid
  misc_feature (57)..(58): Xaa can be any naturally occurring amino acid
  misc_feature (60)..(60): Xaa can be any naturally occurring amino acid
  misc_feature (63)..(64): Xaa can be any naturally occurring amino acid
  misc_feature (76)..(76): Xaa can be any naturally occurring amino acid
  misc_feature (79)..(80): Xaa can be any naturally occurring amino acid
  misc_feature (100)..(100): Xaa can be any naturally occurring amino acid
  misc_feature (107)..(107): Xaa can be any naturally occurring amino acid
  misc_feature (114)..(114): Xaa can be any naturally occurring amino acid
  misc_feature (119)..(119): Xaa can be any naturally occurring amino acid
  misc_feature (122)..(123): Xaa can be any naturally occurring amino acid
  misc_feature (133)..(133): Xaa can be any naturally occurring amino acid
  misc_feature (135)..(135): Xaa can be any naturally occurring amino acid
  misc_feature (148)..(148): Xaa can be any naturally occurring amino acid
  misc_feature (158)..(158): Xaa can be any naturally occurring amino acid
MARTKHXXXRXXXRNXBAXAXXSXAXAAGPXXTPTRRGGKXGXXXQXTTPAATSATXXRXGGXXRSAQAMPSVSKXKKXXRYRPGTVALREIRHFQKTTXLLIPAAXFIREVRXITHFXAPXXVTRWTAEALXAXQEAAEDFLVGLFXDAMLCAIHAXRVTLMRKDFELARRLGGKGRPW

SEQ ID NO: 54 - DNA, 324 bp, Unknown
  consensus HFD nucleotide sequence from A. thaliana, B. rapa, S. lycopersicum and Z. mays
ggctcacagaagaagtcttatcgatacaggccaggaaccgttgctctaaaagagattcgccatttccagaagcagacaaaccttcttattccggctgccagtttcataagagaactgagaagtataacccatatgttggcccctccccaaatcaatcgttggacagctgaagctcttgttgctcttcaagaggcggcagaagattacttggttggtttgttctcagattcaatgctctgtgctatccatgcaagacgtgttactctaatgagaaaagactttgaacttgcacgccggcttggaggaaaaggcagaccatggtga

SEQ ID NO: 55 - protein, 107 aa, Unknown
  consensus HFD polypeptide sequence from A. thaliana, B. rapa, S. lycopersicum and Z. mays
GSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGKGRPW

(19) United States
(12) Patent Application Publication
(10) Pub. No.: US 2018/0116141 A1
(43) Pub. Date: May 3, 2018

(54) HAPLOID INDUCTION

(71) Applicant: The Regents of the University of California, Oakland, CA (US)

(72) Inventors: Sundaram Kuppu, Oakland, CA (US); Anne B. Britt, Oakland, CA (US); Simon Chan (Deceased), Davis, CA (US)

(21) Appl. No.: 15/552,186

(22) PCT Filed: Feb. 23, 2016
(86) PCT No.: PCT/US16/19170

§ 371 Date: Aug. 18, 2017

Related U.S. Application Data
(60) Provisional application No. 62/120,274, filed on Feb. 24, 2015.

(51) Int. Cl. A01H 1/08; A01H 1/02; C07K 14/415; C12Q 1/6895; C12N 15/82

(52) U.S. Cl. CPC A01H 1/08; A01H 1/02; C12N 15/8201; C12Q 1/6895; C07K 14/415

(57) ABSTRACT

Mutations in CENH3 have been identified that are useful for generating haploid progeny.

### CROSS-REFERENCE TO RELATED PATENT APPLICATIONS

The present application claims benefit of priority to U.S. Provisional Patent Application No. 62/120,274, filed Feb. 24, 2015, which is incorporated by reference.

### BACKGROUND OF THE INVENTION

Typical breeding of diploid plants relies on screening numerous plants to identify novel, desirable characteristics. Large numbers of progeny from crosses often must be grown and evaluated over several years in order to select one or a few plants with a desired combination of traits. Hybrid crops are generally produced as the immediate progeny of a cross between two inbred lines. These hybrids express exceptional characteristics derived from both parental genomes, but cannot be further propagated, as the various beneficial alleles segregate during meiosis, resulting in the loss of many of the hybrid's beneficial traits in the next generation. The production of hybrids relies on the production of elite true-breeding parental lines, each homozygous at all loci. These true-breeding lines are usually produced through the repeated self-pollination of an original more heterozygous stock, and are referred to as inbred lines. The production of these elite inbreds normally requires several generations.

The plant breeding process can be accelerated by producing haploid plants, the chromosomes of which can be doubled using colchicine or other means. Such doubled haploids produce homozygous lines in a single generation, which is significantly shorter than the approximately 8-10 generations of inbreeding that is typically required for diploid breeding. Thus, methods of producing haploid plants that can be doubled to generate fertile doubled haploids can dramatically improve the efficiency and effectiveness of plant breeding by producing true-breeding (homozygous) lines in only one generation.

Certain methods of inducing haploid plants by manipulating CENH3 have been described. For example, U.S. Pat. No. 8,618,354 describes introducing recombinant “tailswap” CENH3 constructs into a cenh3 plant to generate a plant (for ease of discussion referred to as a “haploid inducer”) that can be crossed to a second plant to generate progeny that had one set of chromosomes derived from the second plant, with no chromosomes derived from the haploid inducer. For example, if the second plant was diploid, at least some progeny of the cross would be haploid. PCT Publication No. WO2014/110274 describes generating haploid inducer plants by expressing a native CENH3 protein from one species in a different plant species. Expression of the first species's CENH3 in the different species was sufficient to allow for apparently normal mitosis, but resulted in some generation of progeny with half the number of chromosomes of the parent plant crossed to the haploid inducer plant.

### BRIEF SUMMARY OF THE INVENTION

In some embodiments, a plant or plant cell is provided comprising a polynucleotide encoding a non-naturally-occurring CENH3 polypeptide, wherein the CENH3 polypeptide comprises at least one amino acid change compared to an otherwise identical naturally occurring CENH3 polypeptide, wherein the at least one amino acid change is selected from the “Predict Not Tolerated” amino acids in supplementary table 2, where the position of the amino acid in the CENH3 polypeptide and in supplementary table 2 is with reference to the corresponding position in SEQ ID NO:10. In some embodiments, the non-naturally-occurring CENH3 polypeptide comprises a C-terminal histone fold domain (HFD) and the at least one amino acid change is in the HFD. In some embodiments, the non-naturally-occurring CENH3 polypeptide differs by only one amino acid from the naturally occurring CENH3 polypeptide. In some embodiments, the at least one (or only 1-2, or only 1-3) amino acid change occurs at a position corresponding to one of the following positions in SEQ ID NO:10: P82, G83, T84, A86, E89, L100, P102, A104, R124, A127, E128, A129, A132, E135, A136, A137, E138, S148, C151, A152, H154, A155, R157, V158, T159, M161, D164, A168, G172, or G173. In some embodiments, the at least one amino acid change is selected from the following: P82S, P82L, G83R, G83E, T84I, A86T, A86V, E89K, L100F, A104V, R124C, R124H, A127V, E128K, A129T, A129V, A132T, A132V, E135K, A136T, A136V, A137V, E138K, C151Y, A152T, A152V, H154Y, A155T, A155V, R157C, R157H, V158I, T159I, M161I, D164N, A168V, G173R, and G173E (SEQ ID NO:51), wherein the position referenced corresponds to SEQ ID NO:10. In some embodiments, the amino acid is encoded by a codon as indicated under “Mutated codon” of Supplementary table 1. In some embodiments, the naturally occurring CENH3 comprises one of SEQ ID NOs:1-50. In some embodiments, the naturally occurring CENH3 is from A. thaliana, B. rapa, S. lycopersicum, Z. mays, Allium, Beta, Brassica, Capsicum, Cichorium, Citrillus, Cucumis, Cucurbita, Daucus, Lactuca, Phaseolus, Raphanus, Solanum, or Spinacia. In some embodiments, when the non-naturally-occurring CENH3 polypeptide is expressed in a cenh3 knockout plant and said knockout plant is crossed with a wildtype plant having 2N chromosomes, at least 0.1% of progeny have N chromosomes. In some embodiments, the plant belongs to the genus of Allium, Beta, Brassica, Capsicum, Cichorium, Citrillus, Cucumis, Cucurbita, Daucus, Lactuca, Phaseolus, Raphanus, Solanum, or Spinacia. In some embodiments, the non-naturally occurring CENH3 polypeptide is the only CENH3 polypeptide expressed in the plant or plant cell. In some embodiments, the plant comprises a heterologous expression cassette, the expression cassette comprising a promoter operably linked to the polynucleotide.

In some embodiments, a plant or plant cell is provided comprising a polynucleotide encoding a non-naturally-occurring CENH3 polypeptide, wherein the CENH3 polypeptide comprises at least one amino acid change compared to an otherwise identical naturally occurring CENH3 polypeptide, wherein the at least one amino acid change corresponds to G83E, P82S, A86T, R124C, A155T, A136T, A127V, A132V, C151Y, P102L, A104T, A127T, A137T, S148T, G172R, or G172E in SEQ ID NO:10 (see SEQ ID NO:51). In some embodiments, the naturally occurring CENH3 comprises one of SEQ ID NOs:1-50. In some embodiments, the naturally occurring CENH3 is from A. thaliana, B. rapa, S. lycopersicum, Z. mays, Allium, Beta, Brassica, Capsicum, Cichorium, Citrillus, Cucumis, Cucurbita, Daucus, Lactuca, Phaseolus, Raphanus, Solanum, or Spinacia. In some embodiments, the plant is from Allium, Beta, Brassica, Capsicum, Cichorium, Citrillus, Cucumis, Cucurbita, Daucus, Lactuca, Phaseolus, Raphanus, Solanum, or Spinacia. In some embodiments, when the non-naturally-occurring CENH3 polypeptide is expressed in a cenh3 knockout plant and said knockout plant is crossed with a wildtype plant having 2N chromosomes, at least 0.1% of progeny have N chromosomes.

Also provided is polynucleotide (optionally isolated) encoding the non-naturally-occurring CENH3 polypeptide as described above or elsewhere herein. In some embodiments, the naturally occurring CENH3 comprises one of SEQ ID NOs:1-50. In some embodiments, the naturally occurring CENH3 is from B. rapa, S. lycopersicum, Z. mays, Allium, Beta, Brassica, Capsicum, Cichorium, Citrillus, Cucumis, Cucurbita, Daucus, Lactuca, Phaseolus, Raphanus, Solanum, or Spinacia. In some embodiments, when expressed in a cenh3 knockout plant and said knockout plant is crossed with a wildtype plant having 2N chromosomes, at least 0.1% of progeny have N chromosomes.

Also provided is an expression cassette comprising a promoter operably linked to the polynucleotide as described above or elsewhere herein. In some embodiments, the promoter is heterologous to the polynucleotide. Also provided is a host cell comprising the polynucleotide as described above or elsewhere herein. Also provided is a plant comprising the polynucleotide as described above or elsewhere herein or the expression cassette as described above or elsewhere herein.

In some embodiments, a polynucleotide (optionally isolated) encoding a non-naturally-occurring CENH3 polypeptide is provided. In some embodiments, the CENH3 polypeptide comprises at least one amino acid change compared to an otherwise identical naturally occurring CENH3 polypeptide, wherein the at least one amino acid change is selected from the “Predict Not Tolerated” amino acids in supplementary table 2, where the position of the amino acid in the CENH3 polypeptide and in supplementary table 2 is with reference to the corresponding position in SEQ ID NO:10.

In some embodiments, the non-naturally-occurring CENH3 polypeptide comprises a C-terminal histone fold domain (HFD) and the at least one amino acid change is in the HFD. In some embodiments, the non-naturally-occurring CENH3 polypeptide differs by only one amino acid from the naturally occurring CENH3 polypeptide.

In some embodiments, the at least one amino acid change occurs at a position corresponding to one of the following positions in SEQ ID NO:10: P82, G83, T84, A86, E89, L100, P102, A104, R124, A127, E128, A129, A132, E135, A136, A137, E138, S148, C151, A152, H154, A155, R157, V158, T159, M161, D164, A168, G172, or G173. In some embodiments, the at least one amino acid change is selected from the following: P82S, P82L, G83R, G83E, T84I, A86T, A86V, E89K, L100F, P102S, P102L, A104T, A104V, R124C, R124C, R124H, A127T, A127V, E128K, A129T, A129V, A132T, A132V, E135K, A136T, A136V, A137T, A137V, E138K, S148T, C151Y, A152T, A152V, H154Y, A155T, A155V, R157C, R157H, V158I, T159I, M161I, D164N, A168V, G172R, G172E, G173R, and G173E, wherein the position referenced corresponds to SEQ ID NO:10 (see SEQ ID NO:51). In some embodiments, the amino acid is encoded by a codon as indicated under “Mutated codon” of Supplementary table 1.

In some embodiments, the naturally occurring CENH3 comprises one of SEQ ID NOs:1-50. In some embodiments, the naturally occurring CENH3 is from A. thaliana, B. rapa, S. lycopersicum, Z. mays, Allium, Beta, Brassica, Capsicum, Cichorium, Citrillus, Cucumis, Cucurbita, Daucus, Lactuca, Phaseolus, Raphanus, Solanum, or Spinacia.

In some embodiments, when expressed in a cenh3 knockout plant and said knockout plant is crossed with a wildtype plant having 2N chromosomes, at least 0.1% of progeny have N chromosomes.

Also provided is an isolated polynucleotide encoding the non-naturally-occurring CENH3 polypeptide. In some embodiments, the CENH3 polypeptide comprises at least one amino acid change compared to an otherwise identical naturally occurring CENH3 polypeptide, wherein the at least one amino acid change corresponds to P102S in SEQ ID NO:10. In some embodiments, the naturally occurring CENH3 comprises one of SEQ ID NOs:1-50. In some embodiments, the naturally occurring CENH3 is from B. rapa, S. lycopersicum, Z. mays, Allium, Beta, Brassica, Capsicum, Cichorium, Citrillus, Cucumis, Cucurbita, Daucus, Lactuca, Phaseolus, Raphanus, Solanum, or Spinacia. In some embodiments, when expressed in a cenh3 knockout plant and said knockout plant is crossed with a wildtype plant having 2N chromosomes, at least 0.1% of progeny have N chromosomes.

Also provided is an expression cassette comprising a promoter (including but not limited to a CENH3 promoter) operably linked to the polynucleotide encoding the non-naturally-occurring CENH3 polypeptide as described above or elsewhere herein.

Also provided is a host cell comprising the polynucleotide encoding the non-naturally-occurring CENH3 polypeptide as described above or elsewhere herein.

Also provided is a plant comprising the polynucleotide encoding the non-naturally-occurring CENH3 polypeptide as described above or elsewhere herein or the expression cassette as described above or elsewhere herein.

In some embodiments, the plant is selected from B. rapa, S. lycopersicum, or Z. mays. In some embodiments, the non-naturally occurring CENH3 polypeptide is the only CENH3 polypeptides expressed in the plant. In some embodiments, the plant comprises a heterologous expression cassette, the expression cassette comprising a promoter operably linked to the polynucleotide.

Also provided is a method of identifying the plant as described above or elsewhere herein. In some embodiments, the method comprises: generating a plurality of mutated plants, and selecting a plant from the plurality that has the at least one amino acid change. In some embodiments, the selecting comprises Targeting Induced Local Lesions In Genomes (TILLING). In some embodiments, the method further comprises crossing the plant to a parent plant and testing progeny of the cross for chromosome number. In some embodiments, the plurality of mutated plants are generated by exposing plants or seeds to ethyl methanesulfonate (EMS) or other mutagen.

Also provided is a method of making progeny with reduced chromosome content. In some embodiments, the method comprises crossing the plant comprising the mutated CENH3 polypeptide as described above or elsewhere herein to a plant having 2N chromosomes; and selecting progeny from the cross that have N chromosomes. In some embodiments, the progeny from the cross that have N chromosomes are haploid. In some embodiments, haploid plants have haploid chromosomes and the method further comprises doubling the haploid chromosomes of a haploid plant to form homozygous doubled haploid plants. Also provided is progeny from the methods described above. In some embodiments, the progeny are haploid plants. Also provided is a homozygous doubled haploid plant made by the method described above. Also provided is a method of crossing the homozygous doubled haploid plant to another plant to generate F1, F2, or subsequent generations of plants.

### Definitions

“Centromeric histone H3” or “CENH3” refers to the centromere-specific histone H3 variant protein (also known as CENP-A). CENH3 is characterized by the presence of a highly variable N-terminal tail domain, which does not form a rigid secondary structure, and a conserved histone fold domain made up of three α-helical regions connected by loop sections. CENH3 is a member of the kinetochore complex, the protein structure on chromosomes where spindle fibers attach during cell division, and is required for kinetochore formation and for chromosome segregation.

An “endogenous” gene or protein sequence, as used with reference to an organism, refers to a gene or protein sequence that is naturally occurring in the genome of the organism.

A polynucleotide or polypeptide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “promoter,” as used herein, refers to a polynucleotide sequence capable of driving transcription of a coding sequence in a cell. Thus, promoters can include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “plant promoter” is a promoter capable of initiating transcription in plant cells. A “constitutive promoter” is one that is capable of initiating transcription in nearly all tissue types, whereas a “tissue-specific promoter” initiates transcription only in one or a few particular tissue types.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid, and hemizygous.

A “transgene” is used as the term is understood in the art and refers to a heterologous nucleic acid introduced into a cell by human molecular manipulation of the cell's genome (e.g., by molecular transformation). Thus, a “transgenic plant” is a plant that carries a transgene, i.e., is a genetically-modified plant. The transgenic plant can be the initial plant into which the transgene was introduced as well as progeny thereof whose genomes contain the transgene. In some embodiments, a transgenic plant is transgenic with respect to the CENH3 gene. In some embodiments, a transgenic plant is transgenic with respect to one or more genes other than the CENH3 gene.

The phrase “nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read through by a polymerase, and/or formation of double-stranded duplexes, and do not significantly alter expression of a polypeptide encoded by that nucleic acid.

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences which may be introduced to provide codon preference in a specific host cell.

The terms “identical” or percent “identity,” in the context of two or more nucleic acids or polypeptide sequences, refer to two or more sequences or subsequences that are the same or have a specified percentage of nucleotides or amino acid residues that are the same, when compared and aligned for maximum correspondence over a comparison window, as measured using one of the following sequence comparison algorithms or by manual alignment and visual inspection. Two nucleic acid sequences or polypeptides are said to be “identical” if the sequence of nucleotides or amino acid residues, respectively, in the two sequences is the same when aligned for maximum correspondence as described below. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

The phrase “substantially identical,” used in the context of two nucleic acids or polypeptides, refers to a sequence that has at least 50% sequence identity with a reference sequence (e.g., any of SEQ ID NOs: 1-50). Alternatively, percent identity can be any integer from 50% to 100%. Some embodiments include at least: 50%, 55%, 60%, 65%, 70%, 75%, 80%, 85%, 90%, 91%, 92%, 93%, 94%, 95%, 96%, 97%, 98%, or 99%, compared to a reference sequence using the programs described herein; preferably BLAST using standard parameters, as described below.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

Algorithms that are suitable for determining percent sequence identity and sequence similarity are the BLAST and BLAST 2.0 algorithms, which are described in Altschul et al. (1990) J Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

An “expression cassette” refers to a nucleic acid construct that, when introduced into a host cell, results in transcription and/or translation of an RNA or polypeptide, respectively.

The phrase “host cell” refers to a cell from any organism. Exemplary host cells are derived from plants, bacteria, yeast, fungi, insects or other animals. Methods for introducing polynucleotide sequences into various types of host cells are known in the art.

A “mutated CENH3 polypeptide” refers to a CENH3 polypeptide that is a non-naturally-occurring variant from a naturally-occurring (i.e., wild-type) CENH3 polypeptide. As used herein, a mutated CENH3 polypeptide comprises one, two, three, four, or more amino acid substitutions relative to a corresponding wild-type CENH3 polypeptide (e.g., including but not limited to any of SEQ ID NOs: 1-50) while retaining the ability of the polypeptide to support mitosis and meiosis in a plant that does not express another CENH3 polypeptide. In this context, a “mutated” polypeptide can be generated by any method for generating non-wild type nucleotide sequences. In some embodiments, a mutated CENH3 polypeptide, when the only CENH3 polypeptide expressed in a plant, causes the plant to be a haploid inducer plant, meaning when the plant is crossed to a second plant, at least 0.1% of progeny have chromosomes only from the second plant.

An “amino acid substitution” refers to replacing the naturally occurring amino acid residue in a given position (e.g., the naturally occurring amino acid residue that occurs in a wild-type CENH3 polypeptide) with an amino acid residue other than the naturally-occurring residue. For example, the naturally occurring amino acid residue at position 83 of the wild-type Arabidopsis CENH3 polypeptide sequence (SEQ ID NO:10) is glycine (G83); accordingly, an amino acid substitution at G83 refers to replacing the naturally occurring glycine with any amino acid residue other than glycine.

An amino acid residue “corresponding to an amino acid residue [X] in [specified sequence]”, or an amino acid substitution “corresponding to an amino acid substitution [X] in [specified sequence]” refers to an amino acid in a polypeptide of interest that aligns with the equivalent amino acid of a specified sequence. Generally, as described herein, the amino acid corresponding to a position of a specified CENH3 polypeptide sequence can be determined using an alignment algorithm such as BLAST. In some embodiments of the present invention, “correspondence” of amino acid positions is determined by aligning to a region of the CENH3 polypeptide comprising SEQ ID NO:10, as discussed further herein. When a CENH3 polypeptide sequence differs from SEQ ID NO:10 (e.g., by changes in amino acids or addition or deletion of amino acids), it may be that a particular mutation associated with haploid inducing activity of a CENH3 mutant will not be in the same position number as it is in SEQ ID NO:10. For example, amino acid position 49 of Arabidopsis CENH3 (SEQ ID NO:10) aligns with amino acid position 13 of S. lycopersicum CENH3 (SEQ ID NO:29), as can be readily illustrated in an alignment of the two sequences (e.g., FIG. 1B). In this example, amino acid position 49 in SEQ ID NO:10 corresponds to position 13 in SEQ ID NO:29.

### BRIEF DESCRIPTION OF THE DRAWINGS

FIG. 1A1-1A4 Alignment analysis of CENH3 from over 60 different plant species. The N-terminal tail is very variable except for few amino acids at its N-terminus. The C-terminal histone fold domain is relatively conserved.

FIG. 1B Alignment analysis of CENH3 from Arabidopsis, B. rapa, S. lycopersicum, and Z. mays. (Consensus=SEQ ID NO:53; A. thaliana=SEQ ID NO:10; B. rapa=SEQ ID NO:50); S. lycoperiscum=SEQ ID NO:29; Z. mays=SEQ ID NO:16)

FIG. 2 Haploid plants produced by genome elimination in crosses of CENH3 point mutants by Ler gl-1. (a): diploid with trichomes and smaller haploid plant without trichomes (circled in red) (b) sterility phenotype of haploids, undeveloped siliques (circled in red). (c): analysis of control diploid nuclei stained with propidium iodide (PI) by flow cytometry. (d): Flow cytometric analysis of PI stained nuclei from glabrous (putative haploid) offspring. (e): FACS of fertile doubled haploids produced by haploids. (f): DAPI stained nuclei of pollen mother cell of diploid plant showing 10 chromocenters. (g): DAPI stained nucleus of pollen mother cell of haploid plants showing 5 chromocenters. Scale bar on (e) and (f)=5 μm.

FIG. 3. Characterization of haploid genotypes using whole-genome sequencing. (a-d) Top panels show the dosage plots for non-overlapping 100 kb bins across all five Arabidopsis chromosomes with the relative dosage indicated on the y-axis. The bottom panels in each section show SNP analysis based on 1 Mb bins with the percentage of Col-0 SNPs plotted. Regions with 100% Ler SNPs will have 0% Col-0 SNPs. Relative locations of centromeres are indicated by a box. A diploid Col/Ler hybrid control (a) is shown along with a Ler haploid (b). Aneuploid haploids such as a haploid with disomic Chr4 (c) and a Chr4 minichromosome (d) are shown here as well.

FIG. 4 Map of CENH3 histone fold domain showing the location of point mutations. Grey ribbon represents the coding sequence; the triplet codon and the single letter amino acids are represented above the ribbon. Pointers on the ribbon represent conserved sites of EMS-inducible point mutation in the HFD. (SEQ ID NOS:54-55)

### DETAILED DESCRIPTION OF THE INVENTION

### I. Introduction

The inventors have discovered that point mutations can be induced in endogenous CENH3 coding sequences to generate haploid inducer plants. For example, a series of point mutations were generated in Arabidopsis CENH3 and a number of these mutations, when introduced into a cenh3 plant, resulted in plants that induced haploids when crossed to a second diploid parent plant. While the CENH3 mutants described herein can be introduced by plant transformation to generate a haploid inducer plant, one advantage of the mutations described herein is that as few as a single point mutation is involved and thus plants expressing endogenous CENH3 can be mutagenized and screened to identify at least one of the described mutations, thereby generating a haploid inducer plant without plant transformation. Indeed, this is demonstrated in the Examples.

### II. CENH3 Mutants

Endogenous Centromeric histone H3 (CENH3) proteins are a well characterized class of proteins that are variants of histone H3 proteins. These specialized proteins, which are specifically associated with the centromere, are essential for proper formation and function of the kinetochore, a multiprotein complex that assembles at centromeres and links the chromosome to spindle microtubules during mitosis and meiosis. Cells that are deficient in CENH3 fail to localize kinetochore proteins and show strong chromosome segregation defects.

CENH3 proteins are characterized by a N-terminal variable tail domain and a C-terminal conserved histone fold domain made up of three α-helical regions connected by loop sections. The CENH3 histone fold domain is conserved between CENH3 proteins from different species. See, e.g., Torras-Llort et al., EMBO J. 28:2337-48 (2009). In contrast, the N-terminal tail domains of CENH3 are highly variable even between closely related species. Histone tail domains (including CENH3 tail domains) are flexible and unstructured, as shown by their lack of strong electron density in the structure of the nucleosome determined by X-ray crystallography (Luger et al., Nature 389(6648):251-60 (1997)). Additional structural and functional features of CENH3 proteins can be found in, e.g., Cooper et al., Mol Biol Evol. 21(9):1712-8 (2004); Malik et al., Nat Struct Biol. 10(11):882-91 (2003); Black et al., Curr Opin Cell Biol. 20(1):91-100 (2008); and Torras-Llort et al., EMBO J. 28:2337-48 (2009).

CENH3 proteins are widely found throughout eukaryotes, and a large number of CENH3 proteins have been identified. See, e.g., SEQ ID NOs:1-50. It will be appreciated that the above list is not intended to be exhaustive and that additional CENH3 sequences are available from genomic studies or can be identified from genomic databases or by well-known laboratory techniques. For example, where a particular plant or other organism species CENH3 is not readily available from a database, one can identify and clone the organism's CENH3 gene sequence using primers, which are optionally degenerate, based on conserved regions of other known CENH3 proteins.

As discussed in the examples, mutations that computer software identified as “not tolerated” were in fact effective to change CENH3 into a haploid inducer allele. Accordingly, in some embodiments, the CENH3 mutations described herein correspond to those listed as “not tolerated” in supplementary table 2. In some embodiments, the mutation is selected from a position in a CENH3 polypeptide corresponding to one of the following positions in SEQ ID NO:10: P82 (including but not limited to P82S or P82L), G83 (including but not limited to G83R or G83E), T84 (including but not limited to T84I), A86 (including but not limited to A86T or A86V), E89 (including but not limited to E89K), L100 (including but not limited to L100F), P102 (including but not limited to P102S or P102L), A104 (including but not limited to A104T or A104V), R124 (including but not limited to R124C, R124C, or R124H), A127 (including but not limited to A127T or A127V), E128 (including but not limited to E128K), A129 (including but not limited to A129T or A129V), A132 (including but not limited to A132T or A132V), E135 (including but not limited to E135K), A136 (including but not limited to A136T or A136V), A137 (including but not limited to A137T or A137V), E138 (including but not limited to E138K), S148 (including but not limited to S148T), C151 (including but not limited to C151Y), A152 (including but not limited to A152T or A152V), H154 (including but not limited to H154Y), A155 (including but not limited to A155T or A155V), R157 (including but not limited to R157C or R157H), V158 (including but not limited to V158I), T159 (including but not limited to T159I), M161 (including but not limited to M161I0, D164 (including but not limited to D164N), A168 (including but not limited to A168V), G172 (including but not limited to G172R or G172E), or G173 (including but not limited to G173R, and G173E). (SEQ ID NO:52)

Mutations corresponding to the above-described positions and changes can be introduced into a CENH3 coding sequence from any species. In some embodiments the mutated CENH3 polypeptide has one of the mutations described herein and is substantially identical to any one of SEQ ID NOs:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, the CENH3 is from a species of plant of the genus Abelmoschus, Allium, Apium, Amaranthus, Arachis, Arabidopsis, Asparagus, Atropa, Avena, Benincasa, Beta, Brassica, Cannabis, Capsella, Cica, Cichorium, Citrus, Citrullus, Capsicum, Carthamus, Cocos, Coffea, Cucumis, Cucurbita, Cynasa, Daucus, Diplotaxis, Dioscorea, Elais, Eruca, Foeniculum, Fragaria, Glycine, Gossypium, Helianthus, Heterocallis, Hordeum, Hyoscyamus, Ipomea, Lactuca, Lagenaria, Lepidium, Linum, Lolium, Luffa, Luzula, Lycopersicon, Malta, Manihot, Majorana, Medicago, Momodica, Musa, Nicotiana, Olea, Oryza, Panicum, Pastinaca, Pennisetum, Persea, Petroselinium, Phaseolus, Physalis, Pinus, Pisum, Populus, Pyrus, Prunus, Raphanus, Saccharum, Secale, Senecio, Sesamum, Sinapis, Solanum, Sorghum, Spinacia, Theobroma, Trichosantes, Trigonella, Triticum, Turritis, Valerianelle, Vitis, Vigna, or Zea. As described below, the resulting mutated CENH3 polypeptide can be expressed in the same plant species from which the CENH3 polypeptide was derived or the mutated CENH3 polypeptide can be expressed in a different species.

As shown in supplementary table 1, a number of the mutations can be introduced by a single base change in the relevant CENH3 codon to induce the mutation in the CENH3 protein. Supplementary table 1 in the last column illustrates the mutated codon that will induce the corresponding mutation listed. All of the codons shown in supplementary table 1 are induced by G→A or C→T mutations, which are the kind of mutation most typically induced by the mutagen ethyl methanesulfonate (EMS), and thus these mutations can readily be generated in an EMS-mutagenized plant population. However, it will be recognized that other mutation methods, as well as site-directed mutagenesis can be used to generate the mutations described herein as desired. Methods for introducing genetic mutations into plant genes and selecting plants with desired traits are well known and can be used to introduce mutations into or to knock out the CENH3 gene. For instance, seeds or other plant material can be treated with a mutagenic insertional polynucleotide (e.g., transposon, T-DNA, etc.) or chemical substance, according to standard techniques. Such chemical substances include, but are not limited to, the following: diethyl sulfate, ethylene imine, ethyl methanesulfonate and N-nitroso-N-ethylurea. Alternatively, ionizing radiation from sources such as, X-rays or gamma rays can be used. Plants having a mutated or knocked-out CENH3 gene can then be identified, for example, by phenotype or by molecular techniques, including but not limited to TILLING methods. See, e.g., Comai, L. & Henikoff, S. The Plant Journal 45, 684-694 (2006).

Mutated CENH3 polypeptides can also be constructed in vitro by mutating the DNA sequences that encode the corresponding wild-type CENH3 polypeptide (e.g., a wild-type CENH3 polypeptide of any of SEQ ID NOs:1-50), such as by using site-directed or random mutagenesis. Nucleic acid molecules encoding the wild-type CENH3 polypeptide can be mutated in vitro by a variety of polymerase chain reaction (PCR) techniques well-known to one of ordinary skill in the art. See, e.g., PCR Strategies (M. A. Innis, D. H. Gelfand, and J. J. Sninsky eds., 1995, Academic Press, San Diego, Calif.) at Chapter 14; PCR Protocols: A Guide to Methods and Applications (M. A. Innis, D. H. Gelfand, J. J. Sninsky, and T. J. White eds., Academic Press, N Y, 1990).

As a non-limiting example, mutagenesis may be accomplished using site-directed mutagenesis, in which point mutations, insertions, or deletions are made to a DNA template. Kits for site-directed mutagenesis are commercially available, such as the QuikChange Site-Directed Mutagenesis Kit (Stratagene). Briefly, a DNA template to be mutagenized is amplified by PCR according to the manufacturer's instructions using a high-fidelity DNA polymerase (e.g., Pfu Turbo™) and oligonucleotide primers containing the desired mutation. Incorporation of the oligonucleotides generates a mutated plasmid, which can then be transformed into suitable cells (e.g., bacterial or yeast cells) for subsequent screening to confirm mutagenesis of the DNA.

As another non-limiting example, mutagenesis may be accomplished by means of error-prone PCR amplification (ePCR), which modifies PCR reaction conditions (e.g., using error-prone polymerases, varying magnesium or manganese concentration, or providing unbalanced dNTP ratios) in order to promote increased rates of error in DNA replication. Kits for ePCR mutagenesis are commercially available, such as the GeneMorph® PCR Mutagenesis kit (Stratagene) and Diversify® PCR Random Mutagenesis Kit (Clontech). Briefly, DNA polymerase (e.g., Taq polymerase), salt (e.g., MgCl2, MgSO4, or MnSO4), dNTPs in unbalanced ratios, reaction buffer, and DNA template are combined and subjected to standard PCR amplification according to manufacturer's instructions. Following ePCR amplification, the reaction products are cloned into a suitable vector to construct a mutagenized library, which can then be transformed into suitable cells (e.g., yeast cells) for subsequent screening (e.g., via a two-hybrid screen) as described below.

Alternatively, mutagenesis can be accomplished by recombination (i.e. DNA shuffling). Briefly, a shuffled mutant library is generated through DNA shuffling using in vitro homologous recombination by random fragmentation of a parent DNA followed by reassembly using PCR, resulting in randomly introduced point mutations. Methods of performing DNA shuffling are known in the art (see, e.g., Stebel, S. C. et al., Methods Mol Biol 352:167-190 (2007)).

Other mutation induction systems, such as genome editing methods, can be used to target mutations in CENH3, having the advantages of increasing the frequency of single and multiple mutations at a defined target site (Lozano-Juste, J., and Cutler, S. R. (2014) Trends in Plant Science 19, 284-287). The sequence-specific introduction of a double stranded DNA break (DSB) in a genome leads to the recruitment of DNA repair factors at the breakage site, which then repair lesion by either the error-prone non-homologous end joining (NHEJ) or homologous recombination (HR) pathways. NHEJ repairs the breaks, but is imprecise and often creates diverse mutations at and around the DSB. In cells in which the HR machinery repairs the DSB, sequences with homology flanking the DSB, including exogenously supplied sequences, can be incorporated at the region of the DSB. DSBs can therefore be leveraged by geneticists to increase the frequency of mutations at defined sites, however intrinsic differences between the relative roles of HR and NHEJ can affect the mutation types at a targets locus. A number of technologies have been developed to create DSBs at specific sites including synthetic zinc finger nucleases (ZFNs), transcription activator-like endonucleases (TALENs) and most recently the clustered regularly interspaced short palindromic repeats (CRISPR)/CRISPR-associated protein 9 (Cas9) system. This system is based on a bacterial immune system against invading bacteriophages in which a complex of 2 small RNAs, the CRISPR-RNA (crRNA) and the trans-activating crRNA (tracrRNA) directs a nuclease (Cas9) to a specific DNA sequence complementary to the crRNA. Using any of these systems, one can create DSBs at pre-determined sites in cells expressing the genome editing constructs. In order for homologous recombination to occur, a DNA cassette homologous to the targeted site must be provided, preferably at a high concentration so that HR is favored or NHEJ. Multiple strategies are conceivable for realizing this, including template delivery using agrobacterium mediated transformation or particle bombardment of DNA templates, and one recently described method uses a modified viral genome to provide the double stranded DNA template. For example, Baltes et al. 2014 (Baltes, N. J., et al. (2014) Plant Cell 26, 151-163) recently demonstrated that an engineered geminivirus that was introduced into plant cells using Agrobacterium mediated transformation could be engineered to produce DNA recombination templates in cells where a ZFN was co-expressed.

In the CRISPR/Cas9 bacterial antiviral and transcriptional regulatory system, a complex of two small RNAs—the CRISPR-RNA (crRNA) and the trans-activating crRNA (tracrRNA)—directs the nuclease (Cas9) to a specific DNA sequence complementary to the crRNA (Jinek, M., et al. Science 337, 816-821 (2012)). Binding of these RNAs to Cas9 involves specific sequences and secondary structures in the RNA. The two RNA components can be simplified into a single element, the single guide-RNA (sgRNA), which is transcribed from a cassette containing a target sequence defined by the user (Jinek, M., et al. Science 337, 816-821 (2012)). This system has been used for genome editing in humans, zebrafish, Drosophila, mice, nematodes, bacteria, yeast, and plants (Hsu, P. D., et al., Cell 157, 1262-1278 (2014)). In this system the nuclease creates double stranded breaks at the target region programmed by the sgRNA. These can be repaired by non-homologous recombination, which often yields inactivating mutations. The breaks can also be repaired by homologous recombination, which enables the system to be used for gene targeted gene replacement (Li, J.-F., et al. Nat. Biotechnol. 31, 688-691, 2013; Shan, Q., et al. Nat. Biotechnol. 31, 686-688, 2013). The CENH3 mutations described in this application can be introduced into plants using the CAS9/CRISPR system.

Accordingly, in some embodiments, instead of generating a transgenic plant, a native CENH3 coding sequence in a plant or plant cell can be altered in situ to generate a plant or plant cell carrying a polynucleotide encoding a CENH3 mutant polypeptide as described herein. The CRISPR/Cas system has been modified for use in prokaryotic and eukaryotic systems for genome editing and transcriptional regulation. The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize the RNA-mediated nuclease, Cas9 in complex with guide and activating RNA to recognize and cleave foreign nucleic acid. Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21.

### III. Nucleic Acids and Cells

The present disclosure also provides for nucleic acids, including isolated nucleic acids, nucleic acid expression cassettes, and expression vectors, that encode the mutated CENH3 polypeptides described herein. Also provided are cells comprising the nucleic acids.

Once a polynucleotide encoding a mutated CENH3 polypeptide is obtained, in some embodiments, it can also be used to prepare an expression cassette for expressing the mutated CENH3 polypeptide in a transgenic plant, directed by a promoter, which can be endogenous (e.g., a CENH3 promoter) or heterologous. Expression of the mutated CENH3 polynucleotides in a genetic background that otherwise does not express other CENH3 proteins, is useful, for example, to make a haploid inducer plant.

Any of a number of means well known in the art can be used to drive mutated CENH3 activity or expression in plants. In some embodiments, to use a polynucleotide sequence for a mutated CENH3 polypeptide in the above techniques, recombinant DNA vectors suitable for transformation of plant cells are prepared. Techniques for transforming a wide variety of higher plant species are well known and described in the technical and scientific literature. See, e.g., Weising et al. Ann. Rev. Genet. 22:421-477 (1988). A DNA sequence coding for the mutated CENH3 polypeptide can be combined with transcriptional and translational initiation regulatory sequences which will direct the transcription of the sequence from the gene in the intended tissues of the transformed plant.

For example, a plant promoter fragment may be employed to direct expression of the mutated CENH3 polynucleotide in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill.

Alternatively, the plant promoter may direct expression of the mutated CENH3 protein in a specific tissue (tissue-specific promoters) or may be otherwise under more precise environmental control (inducible promoters).

If proper protein expression is desired, a polyadenylation region at the 3′-end of the coding region should be included. The polyadenylation region can be derived from a naturally occurring CENH3 gene, from a variety of other plant genes, or from T-DNA.

In some embodiments, the vector comprising the sequences (e.g., promoters or CENH3 coding regions) comprises a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or Basta.

In some embodiments, the mutated CENH3 nucleic acid sequence is expressed recombinantly in plant cells. A variety of different expression constructs, such as expression cassettes and vectors suitable for transformation of plant cells, can be prepared. Techniques for transforming a wide variety of higher plant species are well known and described in the technical and scientific literature. See, e.g., Weising et al. Ann. Rev. Genet. 22:421-477 (1988). A DNA sequence coding for a CENH3 protein can be combined with cis-acting (promoter) and trans-acting (enhancer) transcriptional regulatory sequences to direct the timing, tissue type and levels of transcription in the intended tissues of the transformed plant. Translational control elements can also be used.

Embodiments of the present invention also provide for a mutated CENH3 nucleic acid operably linked to a promoter which, in some embodiments, is capable of driving the transcription of the CENH3 coding sequence in plants. The promoter can be, e.g., derived from plant or viral sources. The promoter can be, e.g., constitutively active, inducible, or tissue specific. In construction of recombinant expression cassettes, vectors, transgenics, of the invention, a different promoters can be chosen and employed to differentially direct gene expression, e.g., in some or all tissues of a plant or animal.

When generating transgenic plants, it will be desirable to ultimately generate a plant that expresses the mutated CENH3 polypeptide but does not express wildtype CENH3. In some embodiments, one can generate a CENH3 mutation in an endogenous gene that reduces or eliminates CENH3 activity or expression, e.g., generating a CENH3 gene knockout. In these embodiments, one can generate an organism heterozygous for the gene knockout or mutation and introduce an expression cassette for expression of the heterologous corresponding mutated kinetochore complex protein into the organism. Progeny from the heterozygote can then be selected that are homozygous for the mutation or knockout but that comprises the recombinantly expressed heterologous mutated kinetochore complex protein. Accordingly, in some embodiments, plants, plant cells or other organisms are provided in which one or both endogenous CENH3 alleles are knocked out or mutated to significantly or essentially completely lack CENH3 activity, i.e., sufficient to induce embryo lethality without a complementary expression of a mutated CENH3 protein as described herein. In plants having more than a diploid set of chromosomes (e.g. tetraploids), all alleles can be inactivated, mutated, or knocked out.

Alternatively, one can introduce the expression cassette encoding a mutated CENH3 protein into an organism with an intact set of endogenous CENH3 alleles and then silence the endogenous CENH3 gene by any way known in the art. As an example, an siRNA or microRNA can be introduced or expressed in the organism that reduces or eliminates expression of the endogenous CENH3.

Ideally, the silencing siRNA or other silencing agent is selected to silence the endogenous CENH3 gene but does not substantially interfere with expression of the mutated CENH3 protein. In situations where endogenous CENH3 is to be inactivated, this can be achieved, for example, by targeting the siRNA to the N-terminal tail coding section, or untranslated portions, or the CENH3 mRNA, depending on the structure of the mutated kinetochore complex protein. Alternatively, the mutated CENH3 protein transgene can be designed with novel codon usage, such that it lacks sequence homology with the endogenous CENH3 protein gene and with the silencing siRNA.

Also provided are host cell(s) comprising a nucleic acid encoding a mutated CENH3 polypeptide as described herein. As discussed above, the cell can comprise an endogenous CENH3 gene that has been mutated (e.g., via EMS) to contain the nucleic acid encoding the mutated CENH3 polypeptide, or the nucleic acid can be heterologous to the cell (for example, the nucleic acid could be transformed into the cell). In the latter case, the nucleic acid can be part of a heterologous expression cassette (e.g., comprising a promoter operably linked to the coding sequence). Exemplary host cells include, for example, prokaryotic (e.g., including but not limited to E. coli) cells or eukaryotic cells, and can for example plant, fungal, yeast, mammalian, insect, or other cells. Also provided as discussed above are plants comprising a nucleic acid encoding a mutated CENH3 polypeptide as described herein.

### IV. Methods of Generating Haploids

Crossing a plant that expresses a mutated CENH3 polypeptide as described herein (e.g., containing one or more mutations corresponding to those described in supplementary tables 1 or 2), and that does not express a wildtype CENH3 polypeptide, either as a pollen or ovule parent, to a plant that expresses an endogenous CENH3 polypeptide will result in at least some progeny (e.g., at least 0.1%, 0.5%, 1%, 5%, 10%, 20% or more) that are haploid and comprise only chromosomes from the plant that expresses the endogenous CENH3 polypeptide. Thus, the present invention allows for the generation of haploid plants having all of its chromosomes from a plant of interest (i.e., the plant expressing the endogenous CENH3 polypeptide) by crossing the plant of interest with a plant expressing the mutated CENH3 polypeptide and collecting and/or selecting the resulting haploid seed.

As noted above, the plant expressing a wild type (e.g., endogenous) CENH3 protein can be crossed as either the male or female parent. One unique aspect of the present invention is that it allows for generation of a plant (or other organism) having only a male parent's nuclear chromosomes and a female parent's cytoplasm with associated mitochondria and plastids, when the mutated CENH3 polypeptide parent is the female parent.

Once generated, haploid plants can be used for a variety of useful endeavors, including but not limited to the generation of doubled haploid plants, which comprise an exact duplicate copy of chromosomes. Such doubled haploid plants are of particular use to speed plant breeding, for example. A wide variety of methods are known for generating doubled haploid organisms from haploid organisms.

Somatic haploid cells, haploid embryos, haploid seeds, or haploid plants produced from haploid seeds can be treated with a chromosome doubling agent. Homozygous double haploid plants can be regenerated from haploid cells by contacting the haploid cells, including but not limited to haploid callus, with chromosome doubling agents, such as colchicine, anti-microtubule herbicides, or nitrous oxide to create homozygous doubled haploid cells.

Methods of chromosome doubling are disclosed in, for example, U.S. Pat. Nos. 5,770,788; 7,135,615, and US Patent Publication No. 2004/0210959 and 2005/0289673; Antoine-Michard, S. et al., Plant Cell, Tissue Organ Cult., Dordrecht, the Netherlands, Kluwer Academic Publishers 48(3):203-207 (1997); Kato, A., Maize Genetics Cooperation Newsletter 1997, 36-37; and Wan, Y. et al., Trends Genetics 77: 889-892 (1989). Wan, Y. et al., Trends Genetics 81: 205-211 (1991), the disclosures of which are incorporated herein by reference. Methods can involve, for example, contacting the haploid cell with nitrous oxide, anti-microtubule herbicides, or colchicine. Optionally, the haploids can be transformed with a heterologous gene of interest, if desired.

Double haploid plants can be further crossed to other plants to generate F1, F2, or subsequent generations of plants with desired traits.

### EXAMPLES

Production of doubled haploids can greatly accelerate plant breeding. Here we report that a variety of point mutations in highly conserved residues of CENH3 result in haploid induction upon crossing with plants carrying wild-type centromere. Because these mutations can be identified in EMS mutagenized populations, this approach provides a nontransgenic methodology for the identification of haploid inducers in crop species.

With increasing human population and a varying environment there is a pressing need to develop new technologies to accelerate plant breeding (1-3). A rate-limiting step in the production of novel varieties is the number of generations required to obtain true-breeding inbred lines (4). CENH3 is a centromere-specific histone 3 variant that epigenetically marks centromeres (5, 6). Earlier research (7) has shown that modification of the Arabidopsis thaliana CENH3 gene can lead to the production of haploids. Specifically, when the N-terminal tail of histone H3.3 was attached to the Histone Fold Domain (HFD) of CENH3 and tagged with GFP (“GFP tailswap”) this construct complemented a knockout allele of the endogenous CENH3, though resulting in a partially sterile dwarf plant. Interestingly, when crossed with a line carrying wild-type centromeres, the chromosomes derived from the transgenic parent were often lost early in embryogenesis, resulting in plants that carry a haploid set of chromosomes derived only from the nontransgenic parent. During subsequent growth these haploids plants often produced doubled haploids, presumably via rare fortuitous meiotic segregation events or through spontaneous doubling of chromosomes during mitosis.

The GFP-tailswap approach is a transgenic technology. Transgenic crops-including crops that lack transgenes but have a transgenic ancestry—are not approved in several parts of the world and the approval process in permissive countries can be prohibitively expensive. We explored the possibility of creating non-transgenic haploid inducers through point mutations in CENH3. Unlike mutations induced via transgenesis, neither naturally-occurring nor chemically-induced mutations are currently regulated (8).

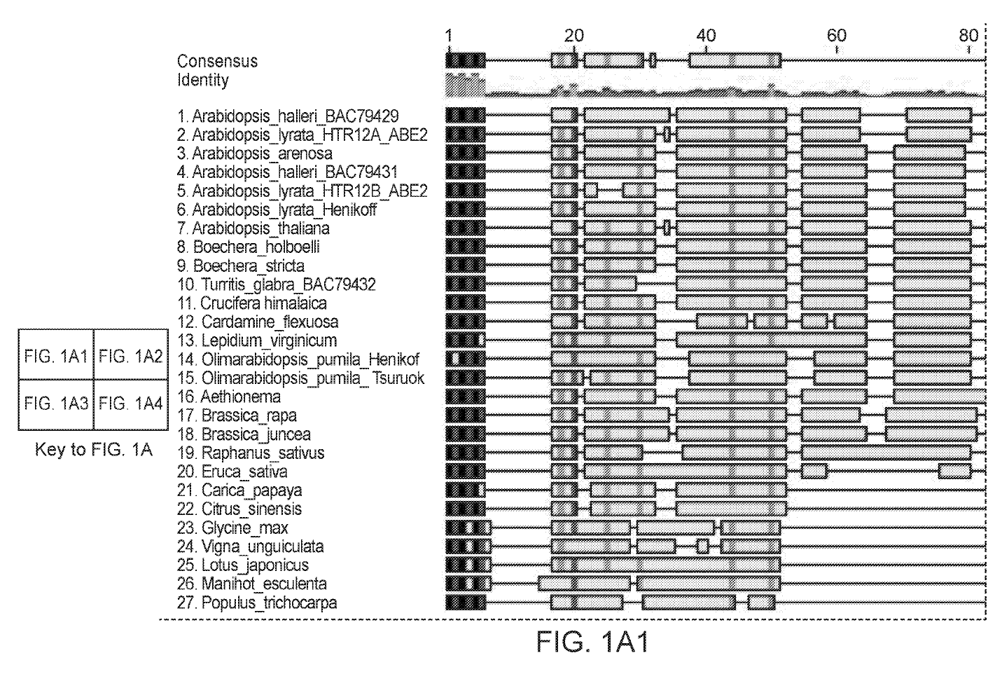

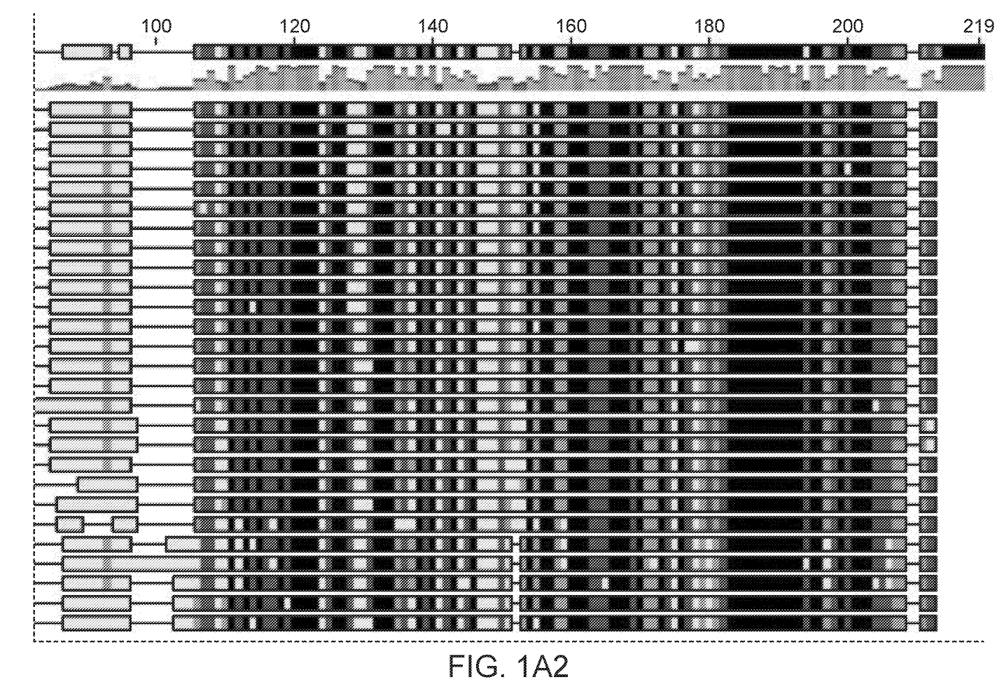

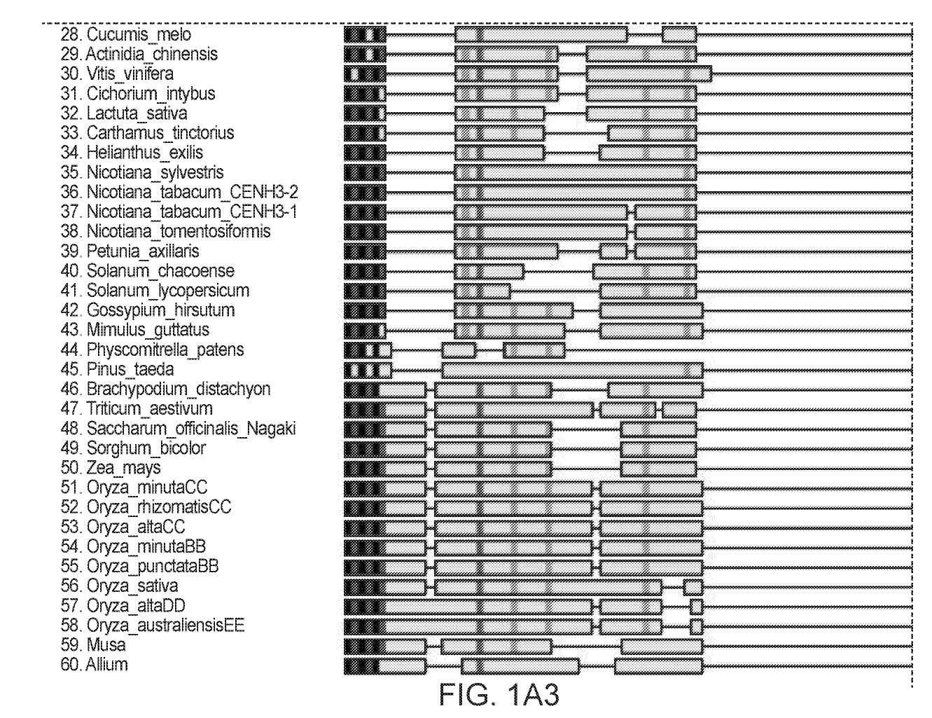

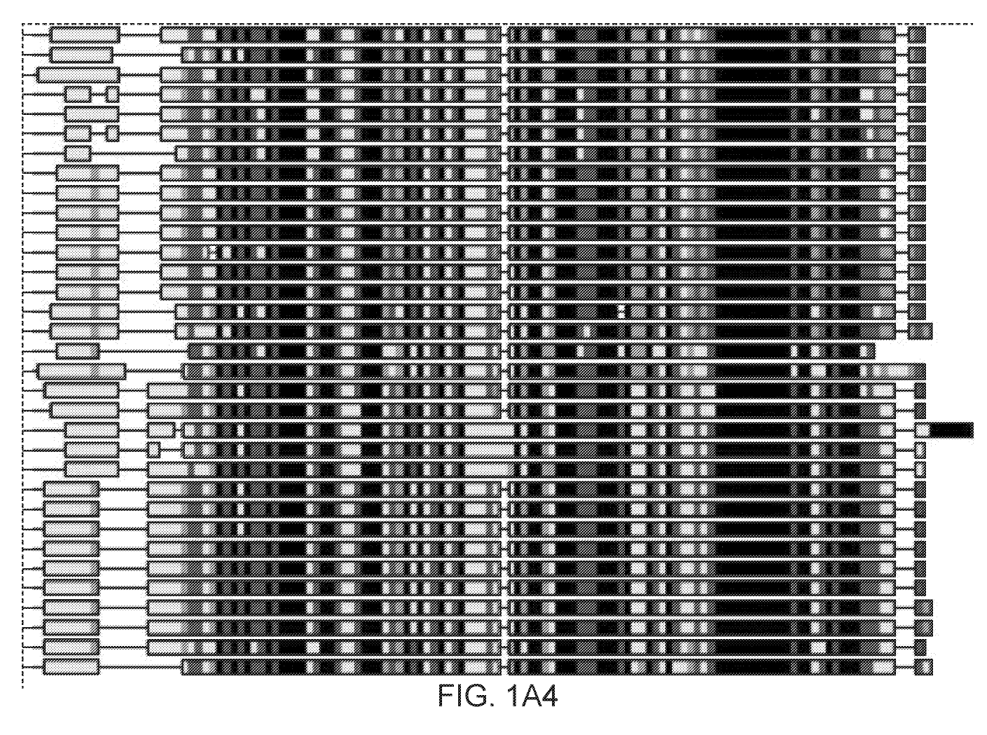

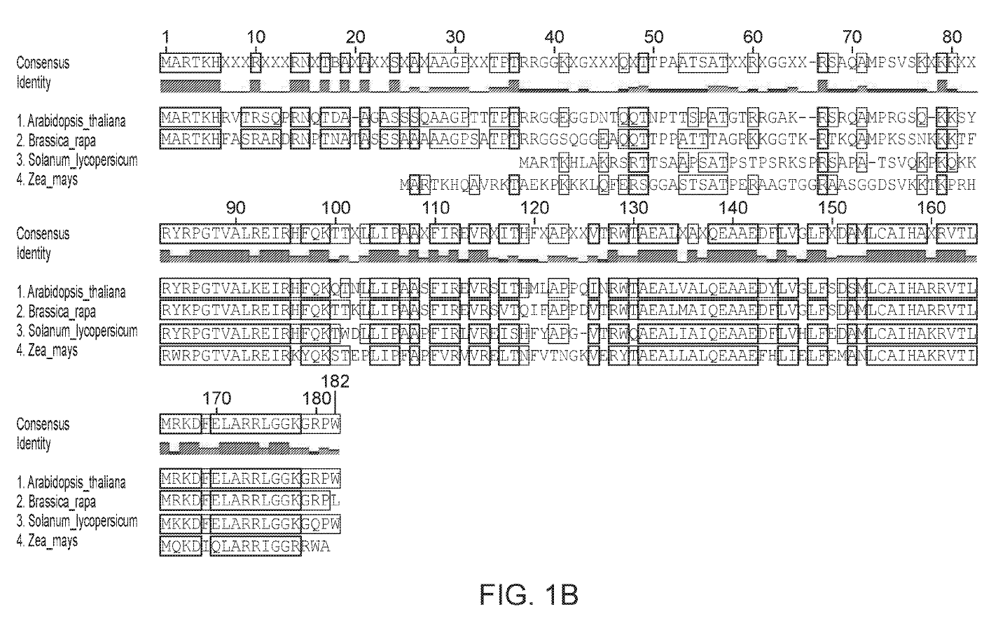

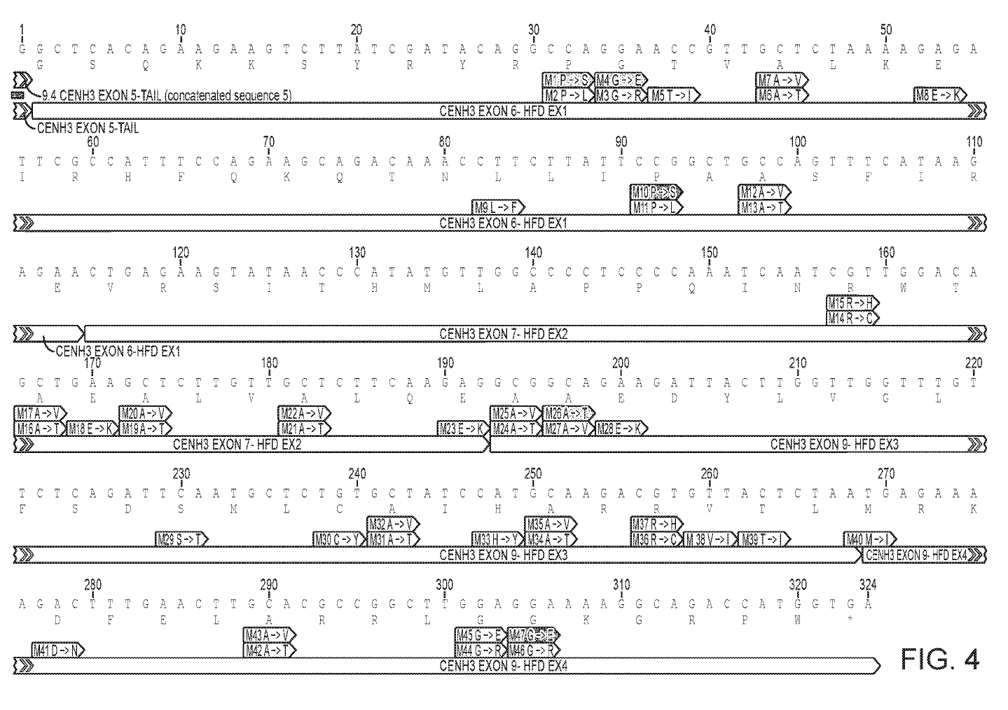

AtCENH3 consists of an N-terminal tail region and a C-terminal histone fold domain (HFD). To identify the conserved domains of CENH3 (and so identify particularly critical amino acids) we aligned the CENH3 protein sequences of over 60 plant species. The tail region is highly variable whereas the HFD is relatively conserved across species (FIG. 1), and for this reason we focused our attention on the HFD. We identified amino acids in Arabidopsis thaliana, Brassica rapa (the progenitor of many crop varieties) Solanum lycopersicum and Zea mays (a monocot) that were conserved and could be mutated to produce the same amino acid change in all four species by G to A or C to T transition (reflecting the mutation spectrum of alkylating chemical mutagens). We identified 47 amino acids in the HFD that fit these criteria (Sup. Table 1 & FIG. 4).

To identify potentially relevant amino acid changes, we used a program (SIFT, http://sift.jcvi.org) (9,10) to predict whether a substitution of one amino acid for another would be functionally tolerated. SIFT predicted that 38 of our candidates would not be tolerated while 9 were more benign (Sup. Table 2). We selected five mutant alleles (Table 1) and tested their ability to transgenically complement a cenh3-1 null mutation (the null allele is zygotic lethal), support fertility, and produce haploids upon crossing by wild-type Arabidopsis. The mutant versions of the gene were synthesized and cloned into a binary vector for agrobacterium-mediated transformation in Arabidopsis (FIG. 4).

To avoid lethality (11), our constructs were transformed into a cenh3-1+/− line and their offspring were screened for both the presence of transgene and native CENH3 genotype. To determine whether alteration in the level of expression of CENH3 (caused by variable levels of expression of the transgene in independently derived transformants) leads to a haploid inducing effect, we generated a wild-type version of our transgene, employing the same vector backbone, native CENH3 promoter, native 5′ UTR and CENH3 tail domain with a synthetic wild-type histone fold domain. Three independent insertion lines carrying WT-HFD were analyzed. In all three lines, WT-HFD was able to complement the nullimorphic cenh3-1 mutation without any obvious phenotypic effect. Upon self-pollination, the plants were fully fertile, did not induce haploids (at the scale measured here, Table 1) and produced 100% normal seeds.

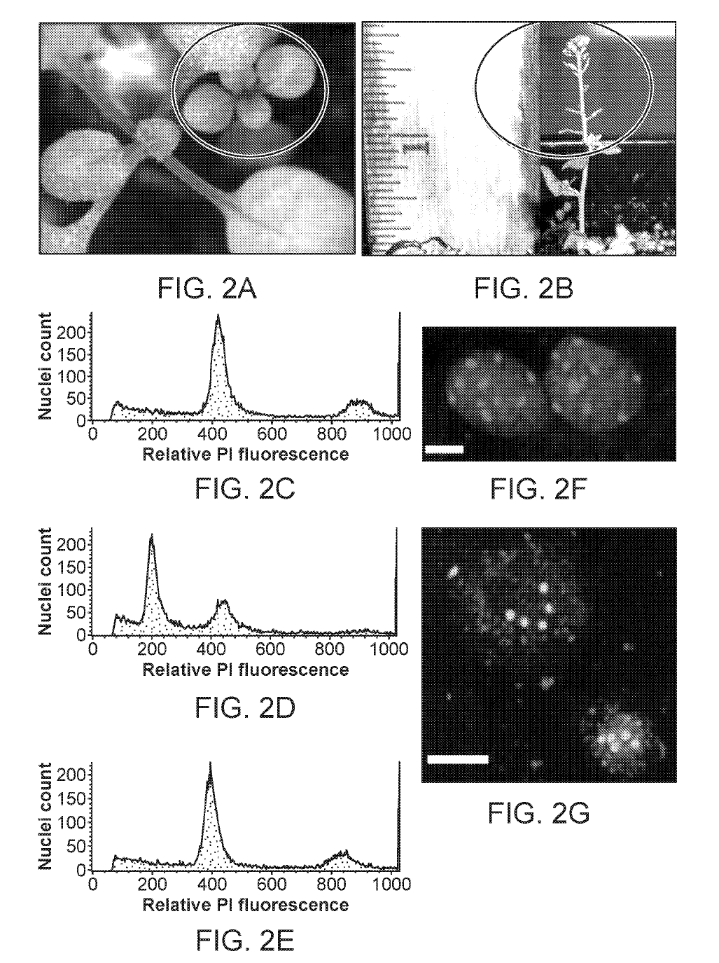

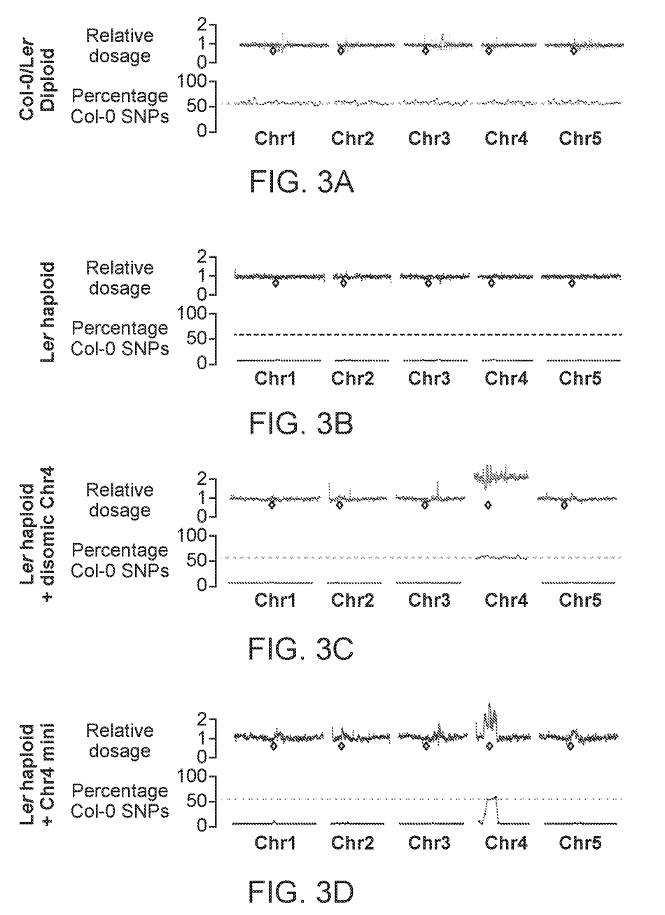

Transgenic plants expressing the single-amino acid substitutions P82S, G83E, P102S, A136T and G173E (Table 1) were viable and fully fertile—thus the mutant transgenes were able to complement the cenh3-1 mutation both mitotically and meiotically. To determine whether the complemented lines were haploid inducers, we crossed them with Landsberg erecta glabrous1 (Ler gl1). These recessive compact and hairless mutations are on chromosome 2 and 3, respectively. We hypothesized that elimination of maternal chromosomes, which carry centromeres decorated with the mutant CENH3, might lead to the production of paternal haploids, which would then exhibit both the erecta and glabrous phenotypes. Crosses of our cenH3−/− plants carrying the WT-HFD transgene by Ler gl-1 pollen produced 100% normal seeds without obvious induction of seed death, a trait associated with haploid induction, and 100% of the progeny were wild-type, suggesting that they were diploids carrying both maternal and paternal chromosomes. The mutant P82S lines, when crossed by the same tester pollen, produced 15-20% dead seeds, and of the viable offspring 2-3% were both erecta and glabrous, consistent with loss of the dominant maternal markers. These putative haploid plants were smaller than corresponding diploids and sterile (FIGS. 2a and b), also consistent with haploidy. Analysis of putative haploids from each point mutant line by flow cytometry confirmed their haploid status (Table 1, FIG. 2 b & c). Similarly, mutants G83E and A136T, while somatically normal and fully fertile on self-pollination, produced both aborted seeds and (flow cytometry-confirmed) haploid progeny, on crossing by Ler gl1-1. Karyotypic analysis of the pollen mother cells confirmed haploid content of 5 chromosomes vs. 10 in diploids (FIG. 2 f & g). Notwithstanding the conservation of these amino acids among angiosperms (Sup. Table 2) and the “not tolerated” prediction by SIFT, the phenotype of plants expressing the altered CENH3 was undistinguishable from wild-type unless crossed by pollen carrying centromeres determined by wild-type CENH3. G173E, another mutation predicted “not tolerated”, appeared to be wild-type even on crossing by wild-type pollen. Similarly, a 5^{th} mutation, P102S, was predicted to be tolerated and indeed seemed to have no effect on CENH3 function.

Next, we performed whole genome sequencing on the resulting haploids to determine their genome contributions. A total of 43 glabrous plants (putative haploids based on phenotyping and flow cytometry) from haploid induction crosses were analyzed. True haploids will appear euploid with no change in the relative copy number of each chromosome. In addition, these chromosomes will carry only paternal sequences (Ler SNPs), in contrast to a true Col-0/Ler diploid from the cross that carries 50% Col-0 SNPs (FIG. 4a). Of the 18 putative haploids from P82S crosses, 15 were clean haploids (FIG. 4b). The remainder of the haploids were Ler plants carrying parts of the Col-0 genome: one was disomic for Chr4 (FIG. 4c), one contained a Chr4 minichromosome (FIG. 4d) and one was disomic Chr4 and also had Chr5 a minichromosome. Analyses of 18 putative haploids from G83E showed that 17 were true Ler haploids except for one, which was a Chr4 disomic. Lastly, all 7 glabrous plants from A136T cross were true Ler haploids.

To determine whether these putative haploids would spontaneously double to produce diploids, we allowed these (nearly sterile) plants to self-pollinate. All haploid plants from the mutants P82S and G83E produced seeds albeit at very low level (20-30 seeds/plant vs. several thousand for wild-type). The seeds were normal in appearance, germinated well and produced glabrous, erecta and fully-fertile offspring. Analysis of ploidy by flow cytometry revealed that the 2C peak of these plants indeed matched the position of the 2C peak of Ler gl-1 (FIG. 2g). These diploid progeny of haploid plants might have arisen via the fortuitous fusion of gametes that were carrying a complete set of five chromosomes each, as has been previously observed in mutants of Arabidopsis in which the gametes segregate without pairing (12).

Technologies to produce doubled haploids (7, 13-16) greatly accelerate plant breeding, but are not available for many crop species. Haploid induction has already been proven helpful in reverse breeding (4), synthetic clonal reproduction (17) and rapid QTL mapping (18) in model plant Arabidopsis thaliana. Though the approach of using an altered, chimeric version of CENH3 (GFP-tailswap) has great potential (19), the transgenic nature of this approach limits its application to crop breeding. A non-transgenic haploid inducer would overcome this shortcoming. Here we show that EMS-inducible point mutants of CENH3 can produce uniparental haploids. These G to A and the C to T transitions are readily identified in existing TILLING (Targeting Induced Local Lesions IN Genomes)(20) populations and so can be immediately applied to crop species. Our analysis suggests that there are 47 conserved (in dicot and monocots), EMS-mutable targets in the CENH3 histone fold domain, of which 38 are predicted by SIFT to be “not tolerated”. Given the frequency at which we identified haploid inducers among the mutations predicted “not tolerated” by SIFT (3 out of 4 tested), our results suggest that all 38 of these mutations (Sup. FIG. 1, Sup. Tablet) are excellent candidates for haploid inducers. Given the fully fertile nature and wild-type growth characteristics of plants carrying these “not tolerated” mutations, all mutations indicated as “not tolerated” are candidates for haploid inducers.

Our transgenic experiments suggest that a large variety of mutations in conserved residues of the CenH3 histone fold domain may result in haploid-inducers that are normal in appearance and fully fertile on self-pollination, while inducing haploids on out-crossing. To confirm haploid inducers exist among mutagenized populations, we analyzed the tilling population generated by Henikoff and Comai available through ABRC (arabidopsis.org). The mutation density of this EMS-treated population was about 3.89 mutations per megabase (Henikoff and Comai, Genome Research, 2003). In a previous screen of approximately 3000 plants from this population, 4 point mutations were found in the histone fold domain. Among these four, one was a silent mutation. The remaining three were A86V, R176K and W178*. Using SIFT, A86V and W178* were predicted to be “not tolerated” and R176K to be tolerated. However, W178 is the last amino acid of CENH3 and on spot-checking this residue did not appear to be conserved. Thus homozygous A86V plants were crossed with Ler gl1. The F1 seeds displayed 32% seed death (a trait which is always found when our haploid inducers are crossed with wild type). We found that 15/110 (13.6%) of the surviving F1 offspring were trichomeless, suggesting that these are paternal haploids. Thus we have shown that haploid inducing lines can be derived without any transgenic manipulation, simply by screening for mutations in conserved residues of the histone fold domain.

Further mutants were generated and tested, with the data from the testing summarized in Table 2. Again, our amino acids in the histone fold domain of CenH3 were mutated to demonstrate that they induce haploidy on crossing with wild-type.

### Materials and Methods

Cloning and Transformation:

Binary vector pCAMBIA-1300 (GenBank: AF234296.1) was used for cloning. The native CENH3 promoter, 5′ UTR and 3′ UTR were cloned into this vector for earlier studies M. Ravi, S. W. L. Chan, Nature 464, 615-618 (2010; M. Ravi et al., Plos Genet 7, (2011).). This clone was used as a starting vector for our study. Cloning was done in three steps. Step 1: CENH3 tail region with introns until first half of intron before HFD was cloned into the KpnI, XbaI site between 5′ and 3′ UTR. Step 2: fragment containing attR1 and attR2 site with CcdB resistance gene was cloned between the CENH3 tail and 3′ UTR into BglI and XbaI site. Step 3: WT-HFD and the point mutants flanked by attL1 and attL2 were synthesized without introns through Genewiz Inc LR recombination was done to obtain the complete CENH3 and transformed into E. coli strain DH5α. The destination vectors were sequenced and transformed into Agrobacterium GV3101 strain and used for Arabidopsis transformation by floral dip method.

Crossing and Analysis of Offspring:

The plants were screened on antibiotic selection for T-DNA carrying point mutation in CENH3 HFD. The antibiotic resistant lines were analyzed for native CENH3 loci by two-step genotyping as described in FIG. 6. Lines carrying transgene with point mutations that were CENH3−/− for the native loci were used as female parent in the crossing. These were crossed with Ler gl-1. The seeds were harvested after three weeks. Offspring were phenotyped for glabrous and erecta traits and subsequently analyzed by flow cytometry and chromosome count.

Flow Cytometry:

Flow cytometric determination of genome content of the wild-type, putative haploids and double haploids were done as described in I. M. Henry et al., Genetics 170, 1979-1988 (2005)

Chromosome Count:

Chromosome count from the pollen mother cell of the wild-type, haploids and double haploids were performed as described in S. J. Armstrong et al., Journal of Cell Science 114, 4207-4217 (2001).

Whole genome sequencing: DNA extraction was done using Nucleon PhytoPure DNA extraction kit (GE Healthcare Life Sciences Inc.). DNA was sheared to 300-400 bp fragments using Covaris E220 sonicator under following settings: Peak incident power 175, duty factor 5%, cycle per burst 200, treatment time 60s at 7° C. Library prep for illumina sequencing was done using standard NEB next DNA Library prep. BIOO Scientific NEXTFlex-96 adapters were used. Samples were pooled and sequenced on MySeq 2500 for 50 bp paired end reads. The resulting reads were further analyzed as described in I. M. Henry et al., Genetics 186, 1231-1245 (2010).

TABLE 1

| | | Amino acid | Aborted seeds | Haploids/Total | |
|---|---|---|---|---|---|
| Line | Codon change | change | (%) | progeny (%) | |
| WT-HFD#1 | No change | No change | 0 | 0/199 | (0) |
| WT-HFD#10 | No change | No change | 0 | 0/243 | (0) |
| WT-HFD#15 | No change | No change | 0 | 0/163 | (0) |
| M1#6 | CCA→TCA | P82S | 15 | 8/334 | (2.4) |
| M1#8 | CCA→TCA | P82S | 21 | 2/20 | (2.7) |
| M1#11 | CCA→TCA | P82S | 20 | 11/435 | (2.5) |
| M4#16 | GGA→GAG | G83E | 36 | 20/164 | (12.2) |
| M4#18 | GGA→GAG | G83E | 28 | 18/197 | (9.1) |
| M10#6 | CCG→TCC | P102S | 10 | 0/203 | (0) |
| M10#19 | CCG→TCC | P102S | 0 | 0/115 | (0) |
| M26#4 | GCA→ACA | A136T | 24 | 7/309 | (2.26) |
| M47#15 | GGA→GAA | G173E | 0 | 0/207 | (0) |

TABLE 2

| | | Amino | | | | |
|---|---|---|---|---|---|---|
| | Codon | acid | Aborted | Hap- | | % |
| Line | change | change | seeds (%) | loids | Total | Haploids |
| M2 | CCA−>CTA | P82L | 6.50% | 2 | 108 | 1.85 |
| M5 | ACC−>ATC | T84I | 0.60% | 0 | 163 | 0 |
| M6 | GCT−>ACT | A86T | 64.30% | 4 | 43 | 9.3 |
| M14 | CGT−>TGT | R124C | 41.50% | 5 | 53 | 9.43 |
| M15 | CGT−>CAT | R124H | 0.50% | 0 | 376 | 0 |
| M17 | GCT−>GTT | A127V | 26.00% | 2 | 111 | 1.8 |
| M21 | GCT−>ACT | A132T | 1.70% | 0 | 146 | 0 |
| M22 | GCT−>GTT | A132V | 33.30% | 2 | 101 | 1.98 |
| M25 | GCG−>GTG | A136V | 37.20% | 1 | 67 | 1.49 |
| M30 | TGT−>TAT | C151Y | 9.80% | 1 | 94 | 1.06 |
| M31 | GCT−> ACT | A152T | 0.70% | 0 | 258 | 0 |
| M32 | GCT−>GTT | A152V | 29.60% | 1 | 41 | 2.44 |
| M34 | GCA-ACA | A155T | 47.92% | 23 | 168 | 13.69 |
| M38 | GTT−>ATT | V158I | 2.20% | 0 | 138 | 0 |
| M44 | GGA−>AGA | G172R | 41.20% | 4 | 64 | 6.25 |

SUPPLEMENTARY TABLE 1
Conserved amino acids across Arabidosis thaliana (SEQ ID NO: 10),
Brassica rapa (SEQ ID NO: 50), Solanum lycopersicum (SEQ ID NO: 29),
and Zea mays (SEQ ID NO: 16) CENH3 histone fold domain that can
be mutated to same amino acid by G to A or C to T transition.
(SEQ ID NO: 51)

| | | | | | Amino acid | Original | Mutated | |
|---|---|---|---|---|---|---|---|---|
| Mutation | A. | B. | S. | Z. | position in | amino | amino | Mutated |
| number | thaliana | rapa | lycopersicum | mays | Arabidopsis | acid | acid | codon |
| 1 | CCA | CCT | CCA | CCA | 82 | P | S | TCA |
| 2 | CCA | CCT | CCA | CCA | 82 | P | L | CTA |
| 3 | GGA | GGA | GGG | GGG | 83 | G | R | AGA |
| 4 | GGA | GGA | GGG | GGG | 83 | G | E | GAA |
| 5 | ACC | ACC | ACA | ACT | 84 | T | I | ATC |
| 6 | GCT | GCC | GCA | GCG | 86 | A | T | ACT |
| 7 | GCT | GCC | GCA | GCG | 86 | A | V | GTA |
| 8 | GAG | GAG | GAA | GAG | 89 | E | K | AAG |
| 9 | CTT | CTT | CTT | CTC | 100 | L | F | TTT |
| 10 | CCG | CCT | CCA | CCC | 102 | P | S | TCG |
| 11 | CCG | CCT | CCA | CCC | 102 | P | L | CTG |
| 12 | GCT | GCC | GCT | GCG | 104 | A | T | ACC |
| 13 | GCC | GCT | GCT | GCG | 104 | A | V | GTC |
| 14 | CCT | CCT | CCT | CGC | 124 | R | C | TGT |
| 15 | CGT | CGT | CGT | CGC | 124 | R | H | CAT |
| 16 | CGT | CGT | CGT | GCA | 127 | A | T | ACT |
| 17 | GCT | GCT | GCT | GCA | 127 | A | V | GTT |
| 18 | GAA | GAA | GAG | GAA | 128 | E | K | AAA |
| 19 | GCT | GCT | GCG | GCC | 129 | A | T | ACT |
| 20 | GCT | GCT | GCG | GCC | 129 | A | V | GTG |
| 21 | GCT | GCT | GCT | GCG | 132 | A | T | ACT |
| 22 | GCT | GCT | GCT | GCG | 132 | A | V | GTT |
| 23 | GAG | GAG | GAG | GAG | 135 | E | K | AAG |
| 24 | GCG | GCG | GCT | GCA | 136 | A | T | ACG |
| 25 | GCG | GCG | GCT | GCA | 136 | A | V | GTG |
| 26 | GCA | GCT | GCT | GCA | 137 | A | T | ACA |
| 27 | GCA | GCT | GCT | GCA | 137 | A | V | GTA |
| 28 | GAA | GAA | GAA | GAA | 138 | E | K | AAA |
| 29 | TCA | GCG | GCA | GCG | 148 | S | T | ACA |
| 30 | TGT | TGC | TGT | TGT | 151 | C | Y | TAT |
| 31 | GCT | GCT | GCT | GCC | 152 | A | T | ACT |
| 32 | GCT | GCT | GCT | GCC | 152 | A | V | GTT |
| 33 | CAT | CAC | CAT | CAT | 154 | H | Y | TAT |
| 34 | GCA | GCA | GCG | GCC | 155 | A | T | ACA |
| 35 | GCA | GCA | GCG | GCC | 155 | A | V | GTA |
| 36 | CGT | CGT | CGT | CGT | 157 | R | C | TGT |
| 37 | CGT | CGT | CGT | CGT | 157 | R | H | CAT |
| 38 | GTT | GTT | GTT | GTC | 158 | V | I | ATT |
| 39 | ACT | ACT | ACA | ACA | 159 | T | I | ATT |
| 40 | ATG | ATG | ATG | ATG | 161 | M | I | ATG |
| 41 | GAC | GAT | GAT | GAC | 164 | D | N | AAC |
| 42 | GCA | GCA | GCT | GCA | 168 | A | T | ACA |
| 43 | GCA | GCA | GCT | GCA | 168 | A | V | GTA |
| 44 | GGA | GGA | GGA | GGA | 172 | G | R | AGA |
| 45 | GGA | GGA | GGA | GGA | 172 | G | E | GAA |
| 46 | GGA | GGA | GGA | GGA | 173 | G | R | AGA |
| 47 | GGA | GGA | GGA | GGA | 173 | G | E | GAA |

Triplet codons and the amino acids of cenH3 histone fold domain from Arabidopsis thaliana, Brassica rapa, Solanum lycopersicum and Zea mays. There are 47 amino acids that could be mutated same amino acid by G to A or C to T transition which can be potentially induced by the chemical mutagen EMS in a non-transgenic way.

SUPPLEMENTARY TABLE 2
SIFT prediction of protein function for substitutions
of amino acids in AtCENH3 (SEQ ID NO: 10)

| | | Seq | |
|---|---|---|---|
| Predict Not Tolerated | Position | Rep | Predict Tolerated |
| y w v t s r q p n l k i h g f e d c a | 1M | 0.58 | M |
| w h y d f n r q m e k c p g l s i | 2A | 0.58 | T V A |
| c w f m d i y v h s p g n l t a e k Q | 3R | 0.58 | R |
| g w y h d r n f q e k c m p s l A I | 4T | 0.58 | V T |
| c w d f m y i g p s h n l a t e q V R | 5K | 0.58 | K |
| c w f m d i y v p g s l a t n e k | 6H | 0.59 | Q R H |
| | 7R | 0.58 | c w d P M e k Q N g R i T s V A h L F Y |
| w h y d f n r q e m k c g s I i | 8V | 0.59 | T P V A |
| w | 9T | 0.59 | c f m y H I P L V g n R Q d T A S K e |
| w f d y i v h g l n s t q P C | 10R | 0.60 | E M A K R |
| w y f c | 11S | 0.61 | m i H v l P G N q R d T A E S K |
| w y f c | 12Q | 0.64 | m h I p v L G d N A T Q E K S R |
| w y f c | 13P | 0.65 | m h i v I q G N T A R e D S P K |
| w y f c m | 14R | 0.68 | H i v P L n G Q D T s E A K R |
| w y f | 15N | 0.67 | c m H i v l P G Q R D N T s A E K |
| w y | 16Q | 0.66 | f c m h i V L G t N D a Q P R S e K |
| w y f c | 17T | 0.60 | h M I l P V G N Q d R S A T e K |
| w y c | 18D | 0.60 | F m h i l V P G R T N Q S A E D K |
| w y f c m h | 19A | 0.62 | v l I n P R D G T S E Q K A |
| | 20A | 0.55 | c w p m D e q k n r G I S H V T f A L Y |
| w y f c | 21G | 0.61 | m h i l V P d T R N S K Q E A G |
| w | 22A | 0.60 | c y m h F i V l P G n R q T d S A K E |
| w y f c m i | 23S | 0.63 | v l g H d P N T Q R K A E S |
| w y f c h | 24S | 0.66 | i M v l q P G D N e T A K R S |
| w y f c m h | 25S | 0.67 | I l n r G q d P V K T E A S |
| w y f | 26Q | 0.64 | C m h i V L r N G P D T S k Q E A |
| w y f c m h | 27A | 0.62 | i l V d Q N P R k T S E G A |
| w y c m h v l | 28A | 0.64 | F n I G P R D Q e T K S A |
| w y f c | 29G | 0.62 | m h I p l V n q D T k E R A S G |
| w y f c m h i v l | 30P | 0.64 | N R D e G A Q S K T P |
| w y f c m h | 31T | 0.66 | i l V n G D R e K P Q A T S |
| w y c m h v l I | 32T | 0.66 | F q G P R D e N K S A T |
| w y f c | 33T | 0.66 | m i H v l g d Q P R N e K A S T |
| w y f c m h i l | 34P | 0.70 | n d V Q G E A T R K S P |
| w y f c | 35T | 0.73 | M H i v l P G n D Q R k A E S T |
| w y f c | 36R | 0.74 | m h i v L P G N D Q S A e T K R |
| w y f c | 37R | 0.77 | m h i l V n P T d G Q A S E K R |
| w y f | 38G | 0.78 | c M H I V L P N R d T G Q S K E A |
| w y c | 39G | 0.78 | F m h I p v L N D Q R T G K A E S |
| w y f | 40E | 0.77 | c m h i V L P G n R Q d T S A K E |
| w y f c m h i | 41G | 0.76 | v l P r N Q D A E K S T G |
| w c f | 42G | 0.76 | Y M i H L V P d N Q R T K S A E G |
| w y f | 43D | 0.78 | c m H I P V l G N R T Q D S A K E |
| w y f | 44N | 0.78 | C m h i v P l G T R Q D N S K E A |
| w y f c | 45T | 0.79 | h M v I L P n G d R Q K E S A T |
| w y f c | 46Q | 0.78 | m h i I V P G n d T R A Q S e K |
| w y f | 47Q | 0.78 | C m h I v l P G n R T d Q S A K E |
| w y f m h i l | 48T | 0.80 | C G q d N P R V E A S K T |
| w y f | 49N | 0.80 | C m h I v L G P R Q N d S T A K E |
| w y f | 50P | 0.82 | C m h I I V G d T N Q A R S K E P |
| w y f c | 51T | 0.82 | m h I P L V n G q d R A E S K T |
| w y f c | 52T | 0.80 | m h i v L P G n d Q R S A K T E |
| w y | 53S | 0.80 | f c M H i v P L G n q R D T A S K E |
| w y f c m h i | 54P | 0.81 | v l n Q D S e R T K A G P |
| w h f y m i c n q d e k | 55A | 0.81 | L P T R V G S A |
| w c | 56T | 0.84 | y f m H i P V L G N Q d R s A e T K |
| c f m y h i l v | 57G | 0.86 | d W k e N Q R P T S A G |
| w y f | 58T | 0.88 | c m h i V L P N G Q R T D A S K E |
| w y f c m | 59R | 0.87 | i H I V P N Q D T A E S G R K |
| w y f c m h | 60R | 0.87 | i v l G N D P T E S Q A R K |
| w c | 61G | 0.80 | m h y F I v L q d P N T K e R S A G |
| W y f | 62A | 0.86 | c m h i L V P G N R Q T D S A K E |
| w y c | 63K | 0.87 | F M H I l V P n G T D R Q A S E K |
| w y | 64R | 0.88 | f c m h I v l P G N T d Q R S A K e |
| w y f c m h | 65S | 0.88 | i L V N d R Q G P E S K T A |
| w y f c | 66R | 0.88 | M H i l V P G N T D Q R S A E K |
| w y | 67Q | 0.88 | c F m H i V L P G N R T D Q S A K E |
| w y f c m | 68A | 0.88 | H I l V N d R G Q K E P T S A |
| w y f | 69M | 0.86 | c M h i P V L G n R T Q D S A K E |
| w f c m i Y v I H | 70P | 0.85 | G N R E D K T Q A S P |
| w v f c | 71R | 0.90 | h M l I V P N G A D T S R K E O |
| w y f c m h | 72G | 0.88 | I l V P D R N K E A Q S T G |
| w y f c | 73S | 0.87 | h M I v L P n d G R Q T A S E K |
| w y f | 74Q | 0.87 | c m H I l P V G N R t D a S Q K E |
| c w f m d y i v s l t A Q P E N G | 75K | 0.95 | H R K |
| c w f d m i y p h n l t a e q G S V | 76K | 0.98 | R K |
| w f c y h i l n d | 77S | 0.98 | M q V e G T S A R K P |
| c w d m g n i e | 78Y | 0.98 | s v q P a l T K F Y R H |
| y w v t s q p n m l k i h g f e d c a | 79R | 1.00 | R |
| c m p q e k r i d t g v a h l S | 80Y | 1.00 | W N F Y |
| c w d f m i y v g p s h n a l t e q | 81R | 1.00 | K R |
| w h y f m i n q r d e k c v t g L S | 82P | 1.00 | A P |
| y w v t s r q p n m l k i h f e d c a | 83G | 1.00 | G |
| w h y f r d q m e c k n g p l i s A V | 84T | 1.00 | T |
| g w h y d n q f s e c p m t I K R A | 85V | 1.00 | L V |
| y w v t s r q p n m l k i h g f e d c | 86A | 1.00 | A |
| y w v t s r q p n m k i h g f e d c a | 87L | 1.00 | L |
| c w d f m i y v g p s h n a l t e Q | 88K | 1.00 | K R |
| y w v t s r q p n m l k i h g f d c a | 89E | 1.00 | E |
| y w v t s r q p n m l k h g f e d c a | 90I | 1.00 | I |
| c w f d m i y v s h g p n l a t e q K | 91R | 1.00 | R |
| c w d m i v g p s Y t l | 92H | 1.00 | F e N A Q H R K |
| h n k r q d g e p c t s a m v i w | 93F | 1.00 | L F Y |
| y w v t s r p n m l k i h g f e d c a | 94Q | 1.00 | Q |
| c w f d m i y v p h g n l t e q S | 95K | 1.00 | A R K |
| w f m y c h i l r v p k d A G E | 96Q | 1.00 | N Q T S |
| | 97T | 1.00 | d m e k q P n C g r i W S h l A F V Y T |
| w c f y m i v l p g t a H | 98N | 1.00 | R S Q K N E D |
| d h g n e c s r k w q t y a v m i F | 99L | 1.00 | P L |
| d h g n e c s w r k y p q t a f m i V | 100L | 1.00 | L |
| h d w n e c p g r q s k y t a f M V | 101I | 1.00 | L I |
| w f m c y d i h n v e t | 102P | 1.00 | g L S Q k A R P |
| | 103A | 1.00 | C w d P m e q n g R I t S K v h A l F Y |
| d h n w y e r c g k q p S | 104A | 1.00 | t f M v I A L |
| w h y f m i r q e d l k n v g C T A | 105S | 1.00 | S P |
| y w v t s r q p n m l k i h g e d c a | 106F | 1.00 | F |
| g w h y d n r f | 107I | 1.00 | k e p C M Q S t L A I V |
| y w v t s q p n m l k i h g f e d c a | 108R | 1.00 | R |
| w | 109E | 1.00 | d p m k n g r s i T h A F C Q E Y V L |
| h w d q n r g e p k c s y f m t a l | 110V | 1.00 | I V |
| c w d f m i y v g s p h n a l t e q | 111R | 1.00 | K R |
| w f y i h l v r p n g t M k C | 112S | 1.00 | A D Q S E |
| h w q d p n e r c g k s y m a f T L | 113I | 1.00 | V I |
| w h f y m r i e l q d k v p n g | 114T | 1.00 | C S A T |
| | 115H | 1.00 | c w p M D E k g Q R N i t S v A H L F Y |
| | 116M | 1.00 | c w M p D G N Q I r k E T S V H A L F Y |
| c w d p e k n q g s | 117L | 0.99 | h a R T M I V Y F L |
| w y f m h | 118A | 0.99 | i C p V N R G d Q T L E K A S |
| w c y h i v M q | 119P | 0.84 | R L N e D F G K A T S P |
| y f c | 120P | 0.84 | W m H i v l G q T N R S P D E A K |
| w f c Y i M | 121Q | 0.98 | H v P G N R A S T L K Q D E |
| c w m k n r t s P h E | 122I | 0.92 | A G F D L Q Y I V |
| w c y | 123N | 0.88 | F m h i p v r Q L D a K G N E T S |
| y w v t s q p n m l k i h g f e d c a | 124R | 1.00 | R |
| k q h n r d g e p c t s a m v i l | 125W | 1.00 | Y F W |
| w f c m y i h l v g p d n a | 126T | 1.00 | r E K S Q T |
| w h y f m i q r n d e c v K | 127A | 1.00 | T L G P S A |
| w c f y m i v l p r g k T | 128E | 1.00 | A N D H S Q E |
| w h y f i m n q r d e l k c v t p G S | 129A | 1.00 | A |
| h d w n e p q c r g s k y t a f m | 130L | 1.00 | V I L |
| h d n e w c p s r k y a G f | 131V | 1.00 | T Q I M V L |
| w h y f i m n q r d e k l v t p g S C | 132A | 1.00 | A |
| d h g n e c w s y r k q t a f M P | 133L | 1.00 | V I L |
| m w f c i y v t l s a p n e d r k g H | 134Q | 1.00 | Q |
| w c m f y i h l v r n g p s k a q d T | 135E | 1.00 | E |
| y w v t s r q p n m l k i h g f e d c | 136A | 1.00 | A |
| w h y f m i r q c e l d k n v p g | 137A | 1.00 | T S A |
| y w v t s r q p n m l k i h g f d c a | 138E | 1.00 | E |
| | 139D | 1.00 | C w p M E k q D n g r I T s v h A l F Y |
| w p d e k q n g r t s i a v l M | 140Y | 1.00 | C H F Y |
| d g h n e c s w y r k p q t a f V M I | 141L | 1.00 | L |
| h w d g n q r y e k s p f m t a l C | 142V | 1.00 | I V |
| w | 143G | 1.00 | f c y m i v l p R T Q a H K S E N G D |
| d h g n e c s w r k y p q t a f V | 144L | 1.00 | M I L |
| h n d k r g e q c p s t a m v w i y | 145F | 1.00 | L F |
| c w m f i y l v r h t p n a k Q G D | 146S | 1.00 | S E |
| w y f c h i p l M t q G N A | 147D | 1.00 | S K R E V D |
| w h y f m i r q d e l n k v p | 148S | 1.00 | C G T S A |
| | 149M | 1.00 | c W p d e q k g r t i s a V H M f l Y N |
| d g n c e s w r k p q t a f v i H Y M | 150L | 1.00 | L |
| k h q e n w r m d s t p y i v f a g L | 151C | 1.00 | C |
| w h y f m i q r d n e c l k v p g T S | 152A | 1.00 | A |
| h d w n e c p g q r s k y t a f m V | 153I | 1.00 | L I |
| y w v t s r q p n m l k i g f e d c a | 154H | 1.00 | H |
| w h y f m i n q r d e l k c v t p s G | 155A | 1.00 | A |
| c w f d m i y v g p s h l a t e q | 156R | 1.00 | N R K |
| y w v t s q p n m l k i h g f e d c a | 157R | 1.00 | R |
| h w d n g q r e y k s p c f m t a l I | 158V | 1.00 | V |
| y w v s r q p n m l k i h g f e d c a | 159T | 1.00 | T |
| h d w n e p c q r g s k y t a f M | 160L | 1.00 | V L I |
| d h n e c s k g r w p q t y a v f i L | 161M | 1.00 | M |
| c w d f m i y v g s | 162R | 1.00 | h n a l e T P R K Q |
| c w f d m i y v g s p h n a l t e q | 163K | 1.00 | R K |
| y w v t s r q p n m l k i h g f e c a | 164D | 1.00 | D |
| d h g n e c s r k p q t y a v W | 165F | 1.00 | L M F I |
| c f m i y l v r g t n s p a k W D | 166E | 1.00 | H E Q |
| d h n g e c s w r k y p q t a f m i V | 167L | 1.00 | L |
| w h y f m i q r n d e c k l v p s g T | 168A | 1.00 | A |
| y w v t s q p n m l k i h g f e d c a | 169R | 0.98 | R |
| y w v t s q p n m l k i h g f e d c a | 170R | 0.98 | R |
| d h g n e c s w r k y p q t a f m v | 171L | 0.98 | L I |
| w f | 172G | 0.98 | m y i h c l q e v d p k n T R S a G |
| y w v t s r q p n m l k i h f e d c a | 173G | 0.97 | G |
| c w f d y | 174K | 0.71 | M v g p s h n a l t q I E R K |
| w h y f i m q c l e n d k v t p s R | 175G | 0.42 | A G |
| c w f d m i y v p s h l n a t e G | 176R | 0.51 | K Q R |
| c w f d m y i v h g n s t a e Q k L | 177P | 0.54 | R P |
| h q k n r d e g p c t s a m v i l y F | 178W | 0.47 | W |

Threshold for intolerance is 0.05. Capital letters indicate amino acids appearing in the alignment, lower case letters result from prediction. ‘Seq Rep’ is the fraction of sequences that contain one of the basic amino acids. A low fraction indicates the position is either severely gapped or unalignable and has little information. Expect poor prediction at these positions.

### REFERENCES

1. Forster, B. P. & Thomas, W. T. B. in Plant Breeding Reviews 57-88 (John Wiley & Sons, Inc., 2010).
2. Wedzony, M. et al. in Advances in Haploid Production in Higher Plants. (eds. A. Touraev, B. Forster & S. M. Jain) 1-33 (Springer Netherlands, 2009).
3. Tester, M. & Langridge, P. Science 327, 818-822 (2010).
4. Wijnker, E. et al. Nat Genet 44, 467-470 (2012).
5. Talbert, P. B. et al. The Plant Cell Online 14, 1053-1066 (2002).
6. Henikoff, S. & Dalal, Y. Current Opinion in Genetics & Development 15, 177-184 (2005).
7. Ravi, M. & Chan, S. W. L. Nature 464, 615-618 (2010).
8. Kharkwal, M. C., and Shu, Q. Y. (ed.) The Role of Induced Mutations in World Food Security Vol. pp. 33-38. (Food and Agriculture Organization of the United Nations, Rome, Italy, 2009).
9. Ng, P. C. & Henikoff, S. Annual Review of Genomics and Human Genetics 7, 61-80 (2006).
10. Kumar, P. et al. Nat. Protocols 4, 1073-1081 (2009).
11. Ravi, M. et al. Genetics 186, 461-471 (2010).
12. Cifuentes, M. et al. PLoS ONE 8, e72431 (2013).
13. Forster, B. P. et al. Trends in Plant Science 12, 368-375 (2007).
14. Chan, S. W. L. Trends in Biotechnology 28, 605-610.
15. Dunwell, J. M. Plant Biotechnol Journal 8, 377-424 (2010).
16. Murovec, J. & Bohanec, B. in Plant Breeding. (ed. I. Abdurakhmonov) (2012).
17. Marimuthu, M. P. A. et al. Science 331, 876 (2011).
18. Seymour, D. K. et al. Proceedings of the National Academy of Sciences, 4227-4232 (2012).
19. Ravi, M. et al. Nature Communications (In Press).
20. Comai, L. & Henikoff, S. The Plant Journal 45, 684-694 (2006).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A plant or plant cell comprising a polynucleotide encoding a non-naturally-occurring CENH3 polypeptide, wherein the CENH3 polypeptide comprises at least one amino acid change compared to an otherwise identical naturally occurring CENH3 polypeptide, wherein the at least one amino acid change is selected from the “Predict Not Tolerated” amino acids in supplementary table 2, where the position of the amino acid in the CENH3 polypeptide and in supplementary table 2 is with reference to the corresponding position in SEQ ID NO:10.

2. The plant or plant cell of claim 1, wherein the non-naturally-occurring CENH3 polypeptide comprises a C-terminal histone fold domain (HFD) and the at least one amino acid change is in the HFD.

3. The plant or plant cell of claim 1 or 2, wherein the non-naturally-occurring CENH3 polypeptide differs by only one amino acid from the naturally occurring CENH3 polypeptide.

4. The plant or plant cell of any of claims 1-3, wherein the at least one amino acid change occurs at a position corresponding to one of the following positions in SEQ ID NO:10: P82, G83, T84, A86, E89, L100, P102, A104, R124, A127, E128, A129, A132, E135, A136, A137, E138, S148, C151, A152, H154, A155, R157, V158, T159, M161, D164, A168, G172, or G173.

5. The plant or plant cell of claim 4, wherein the at least one amino acid change is selected from the following: P82S, P82L, G83R, G83E, T84I, A86T, A86V, E89K, L100F, A104V, R124C, R124H, A127V, E128K, A129T, A129V, A132T, A132V, E135K, A136T, A136V, A137V, E138K, C151Y, A152T, A152V, H154Y, A155T, A155V, R157C, R157H, V158I, T159I, M161I, D164N, A168V, G173R, and G173E, wherein the position referenced corresponds to SEQ ID NO:10.

6. The plant or plant cell of claim 5, wherein the amino acid is encoded by a codon as indicated under “Mutated codon” of Supplementary table 1.

7. The plant or plant cell of any of claims 1-6, wherein the naturally occurring CENH3 comprises one of SEQ ID NOs:1-50.

8. The plant or plant cell of any of claims 1-6, wherein the naturally occurring CENH3 is from A. thaliana, B. rapa, S. lycopersicum, Z. mays, Allium, Beta, Brassica, Capsicum, Cichorium, Citrillus, Cucumis, Cucurbita, Daucus, Lactuca, Phaseolus, Raphanus, Solanum, or Spinacia.

9. The plant or plant cell of any of claims 1-8, wherein, when the non-naturally-occurring CENH3 polypeptide is expressed in a cenh3 knockout plant and said knockout plant is crossed with a wildtype plant having 2N chromosomes, at least 0.1% of progeny have N chromosomes.

10. The plant or plant cell of any of claims 1-9, wherein the plant belongs to the genus of Allium, Beta, Brassica, Capsicum, Cichorium, Citrillus, Cucumis, Cucurbita, Daucus, Lactuca, Phaseolus, Raphanus, Solanum, or Spinacia.

11. The plant or plant cell of any of claims 1-10, wherein the non-naturally occurring CENH3 polypeptide is the only CENH3 polypeptide expressed in the plant or plant cell.

12. The plant or plant cell of any of claims 1-10, wherein the plant comprises a heterologous expression cassette, the expression cassette comprising a promoter operably linked to the polynucleotide.

13. A plant or plant cell comprising a polynucleotide encoding a non-naturally-occurring CENH3 polypeptide, wherein the CENH3 polypeptide comprises at least one amino acid change compared to an otherwise identical naturally occurring CENH3 polypeptide, wherein the at least one amino acid change corresponds to G83E, P82S, A86T, R124C, A155T, A136T, A127V, A132V, C151Y, P102L, A104T, A127T, A137T, S148T, G172R, or G172E in SEQ ID NO:10.

14. The plant or plant cell of claim 13, wherein the naturally occurring CENH3 comprises one of SEQ ID NOs:1-50.

15. The plant or plant cell of claim 13, wherein the naturally occurring CENH3 is from A. thaliana, B. rapa, S. lycopersicum, Z. mays, Allium, Beta, Brassica, Capsicum, Cichorium, Citrillus, Cucumis, Cucurbita, Daucus, Lactuca, Phaseolus, Raphanus, Solanum, or Spinacia.

16. The plant or plant cell of claim 13, wherein the plant is from Allium, Beta, Brassica, Capsicum, Cichorium, Citrillus, Cucumis, Cucurbita, Daucus, Lactuca, Phaseolus, Raphanus, Solanum, or Spinacia.

17. The plant or plant cell of claim 13, wherein, when the non-naturally-occurring CENH3 polypeptide is expressed in a cenh3 knockout plant and said knockout plant is crossed with a wildtype plant having 2N chromosomes, at least 0.1% of progeny have N chromosomes.

18. A polynucleotide encoding the non-naturally-occurring CENH3 polypeptide of any of claims 1-16.

19. The polynucleotide of claim 18, wherein the naturally occurring CENH3 comprises one of SEQ ID NOs:1-50.

20. The polynucleotide of claim 18, wherein the naturally occurring CENH3 is from B. rapa, S. lycopersicum, Z. mays, Allium, Beta, Brassica, Capsicum, Cichorium, Citrillus, Cucumis, Cucurbita, Daucus, Lactuca, Phaseolus, Raphanus, Solanum, or Spinacia.

21. The polynucleotide of claim 18, wherein, when expressed in a cenh3 knockout plant and said knockout plant is crossed with a wildtype plant having 2N chromosomes, at least 0.1% of progeny have N chromosomes.

22. An expression cassette comprising a promoter operably linked to the polynucleotide of any of claims 1-21.

23. The expression cassette of claim 22, wherein the promoter is heterologous to the polynucleotide.

24. A host cell comprising the polynucleotide of any of claims 1-21 or the expression cassette of claim 22.

25. A plant comprising the polynucleotide of any of claims 1-21 or the expression cassette of claim 22.

26. A method of identifying the plant of any of claim 1-17 or 25, the method comprising,
generating a plurality of mutated plants, and
selecting a plant from the plurality that has the at least one amino acid change.

27. The method of claim 26, wherein the selecting comprises Targeting Induced Local Lesions In Genomes (TILLING).

28. The method of claim 26, further comprising crossing the plant to a parent plant and testing progeny of the cross for chromosome number.

29. The method of any of claims 26-28, wherein the plurality of mutated plants are generated by exposing plants or seeds to ethyl methanesulfonate (EMS) or other mutagen.

30. A method of making progeny with reduced chromosome content, the method comprising
crossing the plant of any of claim 1-17 or 25 to a plant having 2N chromosomes; and
selecting progeny from the cross that have N chromosomes.

31. The method of claim 30, wherein the progeny from the cross that have N chromosomes are haploid plants.

32. The method of claim 31, wherein haploid plants have haploid chromosomes and the method further comprises doubling the haploid chromosomes of a haploid plant to form homozygous doubled haploid plants.

33. Progeny made by the method of any of claim 30 or 31.

34. The progeny of claim 33, wherein the progeny are haploid plants.

35. A homozygous doubled haploid plant made by the method of claim 32.

36. A method of crossing the homozygous doubled haploid plant of claim 35 to another plant to generate F1, F2, or subsequent generations of plants.
